(19) United States
(12) Patent Application Publication
(10) Pub. No.: US 2012/0276122 A1
(43) Pub. Date: Nov. 1, 2012

(54) METHODS FOR DIAGNOSIS AND TREATMENT OF CELLULAR PROLIFERATIVE DISORDERS

(75) Inventor: Ze'ev Ronai, San Diego, CA (US)

(73) Assignee: Sanford-Burnham Medical Research Institute, La Jolla, CA (US)

(21) Appl. No.: 13/286,080

(22) Filed: Oct. 31, 2011

Related U.S. Application Data
(60) Provisional application No. 61/408,913, filed on Nov. 1, 2010.

(51) Int. Cl. A61K 39/395; G01N 21/64; C07K 16/18; C12Q 1/68; G01N 33/53; A61P 35/00; G01N 33/573; C40B 30/06

(52) U.S. Cl. 424/174.1; 435/7.4; 506/10; 435/6.13; 435/7.1; 530/389.8; 530/388.2

(57) ABSTRACT

Methods for the diagnosis or prognosis of cellular proliferative disorders by detecting expression of PKC in cancer cells or tumor cells are provided herein. Also provided are methods for treating a melanocyte proliferative disorder with agents that modulate the translocational activity of ATF2 and/or PKCε activity.

### CROSS REFERENCE TO RELATED APPLICATION(S)

This application claims the benefit of priority under 35 U.S.C. §119(e) of U.S. Ser. No. 61/408,913, filed Nov. 1, 2010, the entire content of which is incorporated herein by reference.

### GRANT INFORMATION

This invention was made in part with government support under NCI Grant Nos. CA099961 and CA051995. The United States government has certain rights in this invention.

### BACKGROUND OF THE INVENTION

1. Field of the Invention

The invention relates generally to cancer and more specifically to diagnosis and treatment of tumors and melanoma through activating transcription factor 2 (ATF2) and PKCε.

2. Background Information

Malignant melanoma, the leading cause of death from skin cancer, exhibits a remarkable resistance to chemotherapy and, therefore, remains an area of significant unmet need in oncology. Current understanding of mechanisms underlying melanoma development and progression has enabled development of specific inhibitors targeting the major signaling pathways deregulated in melanoma, including B-Raf, PI3K, and MEK. Although clinical trials of inhibitors for B-Raf and MEK have shown promising results against melanoma, the development of resistance to these monotherapies presents a barrier to the success of these efforts. Best illustrated is the recent example of monospecific therapies, targeting B-Raf , the most prevalent mutation in melanoma. While noting an unprecedented initial response, 100% of tumors recurred within a relatively short time due to subsequent resistance of the tumors to the specific B-Raf inhibitors.

Activating transcription factor 2 (ATF2), a member of the basic HLH family of transcription factors, is one of sixteen Atf/Creb family transcription factors and an integral component of the Activator Protein-1 (AP-1) transcriptional complex, which regulates normal cellular growth and development as well as cellular response to stress. ATF2 is activated in response to stress and cytokine stimuli via phosphorylation by either JNK or p38. Dimerization of ATF2 with other members of the AP1 family results in transcriptional activation of genes involved in regulation of stress, DNA damage, growth, differentiation and apoptosis. ATF2 has been implicated in cellular response to stress and DNA damage, through its transcriptional activity and role in intra-S checkpoint control, through JNK and ATM-dependent phosphorylation, respectively.

The diverse transcriptional functions of ATF2 are attributed to its homo- or heterodimerization with other members of the AP-1 transcription factor family via a basic leucine zipper (bZIP) domain, in concert with its phosphorylation by the stress kinase JNK or by p38 on residues 69/71. As a stress inducible transcription factor, ATF2 regulates gene expression programs implicated in cell cycle control, cytokine expression and cell death. In addition to its transcriptional role, ATF2 functions in cellular response to DNA damage, which require ATM-dependent phosphorylation on residues 490/498. Reports indicate that mice harboring mutations within the ATM phosphoacceptor sites are more sensitive to radiation and exhibit greater genomic instability when crossed with p53 mutant mice or when subjected to a skin carcinogenesis protocol.

Intriguingly, while the above functions require nuclear localization, growing evidence points to cytoplasmic localization of ATF2, although its function in this cellular compartment remains elusive. In melanomas, nuclear ATF2 is associated with metastasis and poor prognosis, whereas cytoplasmic ATF2 was associated with better prognosis. Further, in non-melanoma skin cancers, ATF2 is predominantly cytosolic. Strikingly, ATF2 transcriptional activity (which by default requires nuclear localization) has been implicated in melanoma development, as demonstrated in the N-Ras/Ink4a mouse melanoma model. Correspondingly, inhibition of ATF2 nuclear localization, accomplished by expression of either 10 or 50 amino acid peptides derived from ATF2, efficiently attenuates melanoma development. In contrast, ATF2 cytosolic localization, as seen in non-malignant skin cancer, is associated with a tumor suppressor role, as inhibiting ATF2 in keratinocytes results in a greater number of skin papillomas that develop more rapidly than do control cells.

Over the past decade an important role for ATF2 in skin cancer development has been defined. Genetic inactivation of ATF2's transcriptional function in keratinocytes has reportedly resulted in a higher number of rapidly developing papillomas. Similarly, inactivation of ATF2 in mammary tissue, promoted mammary tumors in a polyoma MT/p53KO model. These findings reveal a tumor suppressor function for ATF2. In contrast, genetic inactivation of ATF2 in melanocytes abolished melanoma formation when mouse mutants were crossed with the mutant N-Ras/Ink4a genetic melanoma model, indicative of its oncogenic role in melanocyte transformation. Several transcription factors can elicit tumor suppressor or oncogenic functions, including Notch and c-myc. Although tissue-specific, the precise underlying mechanisms for the opposing activities of these transcription factors remain largely unknown.

The factors underlying the ability of ATF2 to elicit diverse nuclear and cytoplasmic functions and its role in the development of melanoma remain obscure. Identification of mechanisms implicated in ATF2 subcellular localization and cytosolic function will offer a framework for understanding the regulation of opposing functions for the same transcription factor.

### SUMMARY OF THE INVENTION

The present invention is based on the seminal discovery of the role of ATF2 at the outer membrane of mitochondria, where it is recruited following genotoxic stress in order to promote apoptosis. The ability of ATF2 to mediate this newly discovered function depends on PKC-activity. Constitutively high levels of PKCε in melanoma, prevent nuclear export of ATF2 and subsequent mitochondrial-dependent tumor suppressor function.

Provided herein is a method for the diagnosis or prognosis of a cellular proliferative disorder in a subject. The method includes obtaining from the subject a biological sample; and determining in the sample the expression level of PKC, wherein an increased level of PKC expression in the sample compared to a healthy control indicates a presence or an increased likelihood of a cellular proliferative disorder, thereby providing a diagnosis or prognosis of the disorder in the subject. In one aspect, the method further includes obtaining, from a subject suffering from a cellular proliferative disorder, a pre-therapeutic treatment sample and a post-therapeutic treatment sample; and determining the expression level of PKC in the samples, wherein a decreased level of PKC in the post-treatment sample compared to the pre-treatment sample is indicative of a positive therapeutic treatment. In one embodiment, the PKC isozyme is PKCε.

Also provided herein is a method of treating a cellular proliferative disorder in a subject by administering to the subject an effective amount of an agent that modulates the translocational activity of ATF2, thereby treating the disorder. In one aspect, the agent promotes or increases the translocational activity of ATF2. In one embodiment, the translocational activity is nuclear export or nuclear exclusion and may further include, for example, cytosolic localization.

A method of treating a cellular proliferative disorder in a subject comprising administering to the subject an effective amount of an agent that modulates activity of PKC, thereby treating the disorder is provided herein. In certain aspects, the agent inhibits expression of PKC. The agent may be a selective inhibitor of PKCε and, for example, the PKCε selective inhibitor may have a selectivity ratio of PKCδ IC₅₀ to PKCε IC₅₀ not less than about 10.

Provided herein is a method for identifying an agent for sensitizing cancer cells or tumor cells to apoptosis. The method includes contacting cancer cells or tumor cells with a test agent and detecting apoptosis induced in the cancer cells or tumor cells contacted with the agent as compared to cells not contacted with the agent, wherein apoptosis in the cells contacted with the agent is indicative of an agent that sensitizes cancer cells or tumor cells. In one aspect, the cancer cells or tumor cells are subjected to genotoxic stress prior to, simultaneous with or following contacting the cells with the test agent. In certain aspects, the genotoxic stress is chemotherapy, radiation therapy, or phototherapy. In one embodiment, the tumor cells are melanocytes.

Also provided herein is a method for diagnosis of or prediction of risk of a cellular proliferative disorder in a subject. The method includes obtaining a biological sample from the subject; contacting the sample with an antibody that recognizes phosphorylated ATF2; and detecting the level of phosphorylated ATF2, wherein an increased level of phosphorylated ATF2 in the sample compared to a healthy control indicates a presence or increased risk of a cellular proliferative disorder, thereby providing a diagnosis of or prediction of risk of the disorder in the subject. A finding of increased phosphorylation at T52 is indicative of increased resistance to genotoxic stress and increased survival of the cell. In one embodiment, the phosphorylated ATF2 is located primarily in the nucleus. An increased level of phosphorylated ATF2 in the nucleus may, for example, be indicative of a likelihood of resistance of the cell to genotoxic stress and cell survival.

Provided herein is an antibody that specifically binds to phosphorylated ATF2 including, but not limited to, polyclonal or monoclonal antibodies. In certain aspects, ATF2 is phosphorylated at T52.

The cellular proliferative disorder of the methods provided herein may be, by way of example, a melanocyte proliferative disorder or melanoma including, but not limited to, metastatic melanoma. In one embodiment, the method further comprises administering vemurafenib (Zelboraf™) when the disorder is melanoma.

### BRIEF DESCRIPTION OF THE DRAWINGS

FIG. 1A) is an image depicting control, 5 μM etoposide-treated (ETO, overnight treatment), UVC-treated (20 J/m²), ionizing radiation-treated (IR, 5Gy), or 40 ng/ml leptomycin B (LMB) and ETO co-treated SCC9 cells grown on coverslips were immunofluorescently stained for HSP60,ATF2 or DNA. Immunostained control and ETO panels represent 89±4% and 64±11%, respectively, of the cells from 3 independent replicate coverslips per condition (n>100 cells counted per replicate). B) is a gel that shows control- or ETO-treated SCC9 cells were harvested for whole cell lysate (W) or biochemically fractionated to cytoplasmic (C) and mitochondrial (M) fractions, and subject to immunoblot analysis with indicated antibodies. C) is a photograph depicting coverslip-grown SCC9 cells that were co-treated with 40 ng/ml leptomycin B (LMB) and ETO overnight, and were subsequently fixed and immunofluorescently stained as indicated. D) is a gel depicting SCC9 cells transfected with the Jun2 promoter-luciferase construct and assayed for luciferase activity prior to (Control) or after ETO treatment. E) is a bar graph depicting luciferase activity of mitochondria (M) purified from ETO-treated SCC9 cells subject to proteolysis protection assay with titrated concentrations of trypsin: 0.2 (1), 0.1 (2), 0.05 (3), 0.025 (4), or 0.0125 (5) μg in the absence or presence of 1% Triton X-100 (TX) detergent. Scale bars represent 10 μm.

FIG. 2A) is a gel that shows control or 5 μM etoposide (ETO, overnight)-treated SCC9 cells were lysed and immediately subject to size exclusion chromatography separation. Every other fraction was subject to immunoblot analysis with indicated antibodies. Fractions are arranged from left to right in order of descending molecular weight, with control protein weight markers indicated above (in kDa). B) is a photograph depicting control- or ETO-treated SCC9 cells crosslinked and lysed. ATF2 (upper) or HK1 (lower) were immunoprecipitated and subject to immunoblot analysis with indicated antibodies. C) is a gel showing coverslip-grown SCC9 cells knocked down for either HK1 or HK2 and subject to ETO treatment and subsequently immunofluorescently stained with indicated antibodies. D) is graph that shows quantitation of mitochondrial ATF2 (MtATF2, grey bars) versus displaced ATF2 (red bars) before or after knockdown of HK1 or HK2, n>50 cells per replicate experiment over 3 independent experiments. Scale bars=10 μm.

FIG. 3A) is an image depicting coverslip-grown SCC9 cells treated with 1 or 5 μM PKCε translocation inhibitor overnight, and immunofluorescently stained with indicated antibodies. B) is a photograph depicting coverslip-grown SCC9 cells treated with 10 nM siRNA targeting PKCε overnight were immunofluorescently stained with indicated antibodies. C) is a photograph depicting coverslip-grown SCC9 cells transfected with HIS-tagged constitutively active (ca) PKCε were immunofluorescently stained with indicated antibodies. D) is a gel showing SCC9 cells treated with 5 μM etoposide (ETO, overnight) or 5 μM PKCε inhibitor (PKCε-i) overnight, lysed and subject to immunoblot analysis with indicated antibodies. F) is a photograph depicting coverslip-grown SCC9 cells were transfected with HA-tagged wildtype ATF2 (WT), ATF2T52A or ATF2T52E, and subsequently immunofluorescently stained with indicated antibodies. G-H) is a series of bar graphs that show quantitation of (Left) SCC9 cells transfected with Jun2 promoter-luciferase construct and empty vector (EV) or caPKCε, assayed for luciferase activity after ETO treatment. (Right) SCC9 cells stably knocked down for ATF2 with a 3′-UTR-targeted shRNA, reconstituted with Jun2 promoter-luciferase construct and ATF2 (WT), ATF2T52A or ATF2T52E and subsequently assayed for luciferase activity. Scale bars represent 10 μm.

FIG. 4 is an image that depicts coverslip-grown SCC9 cells transfected with empty vector (EV) or HA-tagged ATF2 (WT), ATF2^{T52A} or ATF2T52E in the absence (A, C, E, E*, G) or presence (B, D, F, F*, H) of 5 μM etoposide (ETO, overnight), and subsequently immunostained with indicated antibodies and MitoTracker. The arrowheads indicate HA-positive cells. Scale bars represent 10 μm.

FIG. 5A) is a series of gels showing SCC9 cells transfected with empty vector (EV) or HA-tagged ATF2 (W), ATF2^{T52A} (A) or ATF2^{T52E} (E) in the presence or absence of 5 μM etoposide (ETO, overnight), crosslinked and lysed for whole cell lysate (input) or immunoprecipitated for VDAC1 and subject to immunoblot analysis with indicated antibodies. B) is a series of gels showing SCC9 cells treated with ETO (E) or PKCε translocation inhibitor (i, 5 μM) or both (i-E), lysed for whole cell lysate (input) or immunoprecipitated for VDAC1 and subject to immunoblot analysis with indicated antibodies. C) is a bar graph depicting SCC9 cells transfected with empty vector (EV), wild-type ATF2 (WT), ATF2^{T52A} or ATF2^{T52E} in the presence (*, dark grey bars) or absence (grey bars) of 5 μM etoposide overnight, pulse labeled with tetramethylrhodamine ethyl ester (TMRE) or nonylacridine orange (NaO), and subsequently analyzed by FACS analysis. Histogram bars represent ratios of TMRE/NaO uptake. n=10,000 cells per replicate; 3 replicates per condition were performed. p<0.0001; #: p=0.01 D) is a photograph depicting coverslip-grown SCC9 cells transfected with empty vector (EV), wild-type ATF2 (WT), ATF2^{T52A} or ATF2^{T52E} immunostained for cytochrome C and MitoTracker, and analyzed for cytochrome C release by fluorescence microscopy (n>100 cells per replicate; 3 replicates per condition were performed). •:p=0.0008 E) Right, is a graphic representation of SCC9 cells treated with PKCε translocation inhibitor (PKCε-i, 5 μM) alone or in the presence or 5 μM etoposide (ETO, overnight) stained with Annexin-V and propidium iodide (PI) and subject to FACS analysis. •: p<0.0001; #: p=0.6018. Representative FACS plots (left) and corresponding Annexin V-negative and Annexin V-positive (AV−, AV+) percentages are as follows: control (97%, 3%); PKCε-i (92%, 8%); ETO (89.5%, 11.5%); and ETO+PKCε-i (87%, 13%). Averaged value histograms (right) are displayed as indicated. F) is a series of bar graphs that show SCC9 cells transfected with empty vector (EV) or HA-tagged ATF2 (WT), ATF2^{T52A} or ATF2^{T52E} in the presence (*, dark grey bars) or absence (grey bars) of ETO. Cells were stained with Annexin-V and subject to FACS analysis. n=10,000 cells per replicate; 3 replicates per condition were performed. •: p=0.0001; #: p<0.0001.

FIG. 6A) is a gel that shows normal human epidermal keratinocytes (NHEK), three SCC cell lines (M7, P7, SCC9 ), five melanoma cell lines (501 Mel, LU1205, WM793, UACC903) and normal human epidermal melanocytes (HEM) harvested and subject to immunoblotting or immunoprecipitation and immunoblotting as indicated. B) is a photograph depicting control or ETO-treated coverslip-grown MeWo, 501Mel or UACC903 cells immunofluorescently stained with indicated antibodies. C) is a series of plots that show 501Mel cells treated with siRNA targeted against PKCε (siPKCε) alone or in the presence or absence of 5 μM etoposide (ETO, overnight) or UVC (5 J/m2) were stained with Annexin-V and propidium iodide (PI) and subject to FACS analysis. Representative FACS plots (upper) and averaged value histograms (lower) are displayed as indicated.

FIG. 7A) is a scatterplot of VDAC1 versus ATF2 expression levels extracted from the Yu et al. global expression profiling study (Yu et al., 2008, J. Invest. Dermatol., 128,1797-1805). Pearson correlation coefficients ‘r’ are shown for the two types of tissue. B) is a graph showing distribution of all expression correlation coefficients for gene pairs in Lo et al. Distributions are shown separately for the normal samples and the BCC samples. The loss of correlation from normal to BCC is significant at a level of p<0.05. C) Scatterplot of VDAC1 versus ATF2 expression levels extracted from an independent expression profiling study by Xu et al. (Xu et al., 2008, Mol. Cancer Res., 6, 760-769). D) is a graph showing distribution of all expression correlation coefficients from the Xu et al. study, with display similar to (B). The loss of correlation from primary to metastatic tumor is significant at a level of p<0.05. E) is a graphic representation of PKCε distribution in metastatic and primary specimens showing significantly higher levels in the metastatic specimens (P<0.0001, t-statistic—4.257). (F) is a plot of Kaplan Meier survival curves showing significantly shorter survival in higher PKCε expressers in primary tumors.

FIG. 8 is a photograph depicting control—or 5 μM etoposide-treated (ETO, overnight) coverslip-grown normal human skin fibroblasts (HSF), immortalized human keratinocytes (HaCaT), or human epidermal melanocytes (HEM) immunostained with the indicated antibodies. Scale bar represents 10 μm.

FIG. 9A) is a gel that shows SCC9 cells transfected for 36 hours with 10 nM siRNA against hexokinase 1 (HK1) or hexokinase 2 (HK2), or control siRNA (SC), harvested and lysates were subject to immunoblot analysis. B) is a photograph showing control or UACC903 cells treated with either 1 μM SP600125 (JNK inhibitor) or with 5 uM SB203580 (p38 inhibitor) for 4 hours, or with 2 μM GÖ6850 for 3 hours were fixed and immunostained for ATF2, phalloidin and DAPI.

FIG. 10A) is a gel showing SCC9 cells transfected with 10 nM scrambled (scr) or PKCε-targeted siRNA. B) is a photograph depicting SCC9 cells either infected with PLKO empty vector (EV) or PLKO-shPKCζ for 72 hours (upper) or transfected with 10 nM scrambled (scr) or PKCδ-targeted siRNAs (lower, 1, 2, 3) and immunofluorescently with indicated antibodies or harvested for immunoblot analysis. C) is a series of gels that show SCC9 cells infected for 48 hours with PLKO EV or shATF2 (shRNA lentivirus targeting ATF2 3′-UTR) and selected with 1 μg/ml puromycin for at least 5 days following infection. Scale bars represent 10 μm.

FIG. 11 A-C) is a graphic representation of cells with empty vector or HA-tagged wild-type ATF2 (WT), T52A or T52E mutant contstructs in the presence (asterix, red bars) or absence (grey bars) of 5 uM etoposide overnight. Cells were then pulse-labelled with (250 nM, 20 min) TMRE (A) or (10 nM, 20 min) Nao (B), and subjected to FACS analysis. C) is a gel showing SCC9 cells transfected as in A and B were used for immunoblot analysis with indicated antibodies. D) is a graph depicting statistical quantitation of HA-tagged ATF2 WT, T52A or T52E mutant-overexpressing cells from FIG. 4, binned by percentage nuclear, nuclear+mitochondrial, or mitochondrial-only (n>50 cells per replica in over 3 independent replicates; asterix indicates cells treated with etoposide). E) is a photograph: magnification of T52A+ETO inset from FIG. 4.

FIG. 12A-C) is a graphic representation showing SCC9 cells treated with PKCs inhibitor alone or after UVC (20 J/m², yellow bars) treatment stained with propidium iodide (PI) and subject to FACS analysis. •:p=0.0008; #: p=0.0055. B) SCC9 cells treated with 20 nM scrambled control (CTL) or PKCε-targeted (siPKCε) siRNA alone or in the presence or absence of 5 μM etoposide (ETO, overnight, red bars) or UVC were stained with PI and subject to FACS analysis. Due to experimental procedures, cells were ˜35% more confluent than panel A when treated with genotoxic stress. •: p<0.0001; #: p=0.001 C) 501 Mel cells treated with CTL or siPKCε alone or in the presence or absence of ETO (overnight, red bars) or UVC were stained with PI and subject to FACS analysis. •: p=0.03; #: p=0.0006 D) is an image of representative panels illustrating control (left) or cytochrome C-leaking cells (right). Briefly, cells transfected as in FIG. 5D were immunostained for COXIV and cytochrome C. Points of cytochrome C leakage from mitochondria are indicated with yellow arrowheads. For FACS analyses, n=10,000 cells per replicate over 3 independent replicates.

FIG. 13A-C)is a graphic representation showing melanoma exhibiting upregulated PKCε are more resistant to genotoxic stress-induced cell death compared to lower expressing counterparts; ATF2-T52E confers resistance to genotoxic stress A) PKCε low-expressing (SCC9 , P9, UACC903) or high-expressing (501 Mel, LU1205) cell lines were grown for 24 hours in the absence (CTL) or presence of 5 or 15 μM etoposide (ETO), stained with PI and subject to FACS analysis. B) SCC9 cells were transfected with either empty vector (EV), wild-type (WT) or T52E-ATF2 (T52E), and subject to control (1), 5 μM etoposide (eto, 2), 10 μM PKCε translocation inhibitor (PKCε-i, 3), PKCε-i+eto (4), 20 J/m² UVC (5), or PKCε-i+UVC (6). Cells were harvested 16 hours after treatment, stained for Annexin-V-FITC, and subject to FACS analysis (n=10,000 cells per replicate over 3 independent replicates). C) SCC9 cells were infected and stably puromycin-selected with either pBabe-EV, -ATF2 -WT, or -ATF2 -T52E. 45,000 cells per line were seeded into 12-well plates, and grown either at non-stressed (left) or in the presence of 5 uM ETO for 8 days (right, ETO was initially added 24 hours post plating; on day 5, media was refreshed and re-supplemented with 5 μM ETO). Cells were harvested at indicated timepoints and subject to staining and FACS analysis for Annexin-V-FITC staining. *: p=0.0072

FIG. 14 A-B) are photographic images. A) Coverslip-grown SCC9 cells were immunostained with the pT52 ATF2 Ab and DAPI, either in the absence of peptide, or the presence of a non-phosphorylated control or phosph-T52 ATF2 peptide, or with secondary antibody alone. B) Representative high and low PKCε expression histospots from large cohort melanoma tumor microarray. C-G) are a series of graphs. C) Kaplan Meier survival curves showing no difference in survival in metastatic melanoma patients whose tumors express higher levels of PKCε. D) Scatterplot of VDAC 1 versus ATF2 expression levels extracted from the Yu et al. global expression profiling study. Red points represent different tissue samples of basal cell carcinoma (BCC), whereas green points represent samples of non-lesional (normal) skin epithelium. Pearson correlation coefficients ‘r’ are shown for the two types of tissue. E) Distribution of all expression correlation coefficients for gene pairs in Lo et al. Distributions are shown separately for the normal samples and the BCC samples. The loss of correlation from normal to BCC is significant at a level of p<0.05. F) Scatterplot of VDAC1 versus ATF2 expression levels extracted from an independent expression profiling study by Xu et al. G) Distribution of all expression correlation coefficients from the Xu et al. study, with display similar to (E). The loss of correlation from primary to metastatic tumor is significant at a level of p<0.05.

FIG. 15 is a table depicting flag-tagged wild-type ATF2 was transfected into SCC9 cells and subsequently immunoprecipitated from cytoplasmic extracts. Immunoprecipitates were subject to mass spectrometric analysis. Mitochondrial outer membrane-related identified proteins were subsorted and arranged according to number of peptides in analysis.

FIG. 16 is a schematic representation of cellular translocational activity of ATF2.

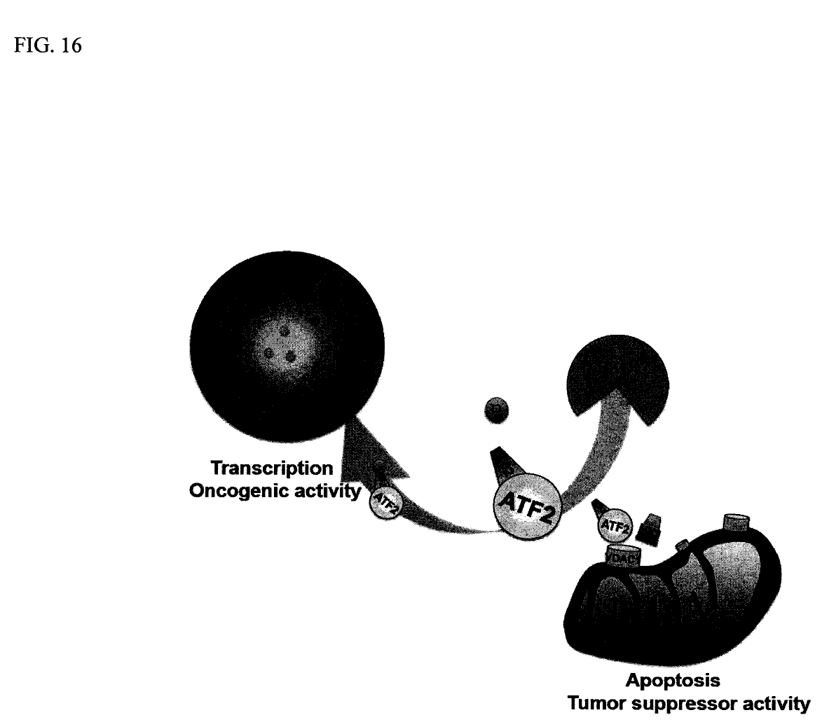

FIG. 17 D) is a gel that depicts ATF2^{T52A} or ATF2^{T52E} overexpressing SCC9 cells were subject to gel filtration and immunoblot analysis with indicated antibodies. E) is a photograph that shows SCC9 cells treated with 5 μM PKCε translocation inhibitor (PKCε-i) alone for 16 hours or with 6 hours of pre-treatment with 40 ng/ml leptomycin B. Scale bars represent 10 μm. F) is a gel showing SCC9 cells treated with 5 uM PKCε translocation inhibitor (PKCμ-i) overnight, fractionated to whole cell lysate (W), cytoplasmic (C) and mitochondrial (M) fractions and subject to immunoblot analysis with indicated antibodies. *: non-specific band.

FIG. 18A-C) are gels. A) SCC9 cells were transfected with empty vector or constitutively active PKCε (caPKCε), and subject to immunoblot analysis with indicated antibodies. A rabbit polyclonal antibody was generated against a phosphorylated Thr52 ATF2 phosphopeptide (PhosphoSolutions, Inc.). Densitometric ratios of bands for (−caPKCε, +caPKCε) are as follows: pS729/β-actin (0.193, 0.636); pT52/Total ATF2 (0.215, 0.662). B) Control or ATF2 -knocked down LU1205 cells were subject to immunoblot analysis with indicated antibodies. C) Indicated cell lines were subject to (10 μM, ETO, overnight) etoposide treatment and subject to immunoblot analysis with indicated antibodies. Densitometric band ratios for pT52/Total ATF2 (−ETO, +ETO) are as follows: SCC9 (1, 0.4), UACC903 (1, 0.7), 501Mel (1, 0.9), LU1205 (1, 1). Densitometric band ratios for pS729/Total PKCε (−ETO, +ETO) are as follows: SCC9 (1, 0.7), UACC903 (1, 0.7), 501Mel (1, 0.97), LU1205 (1, 1). D) is an image showing control or ETO-treated coverslip-grown LU1205, 501Mel or UACC903 cells immunofluorescently stained with indicated antibodies. E) is a graphic representation depicting 501Mel cells treated with 10 μM PKCε translocation inhibitor (PKCε-i) alone or in the presence or absence of 5 μM etoposide (ETO, overnight) were stained with Annexin-V and subject to FACS analysis. n=10,000 cells per replicate; 3 replicates per condition were performed. •: p32 0.0009; #: p=0.0032.

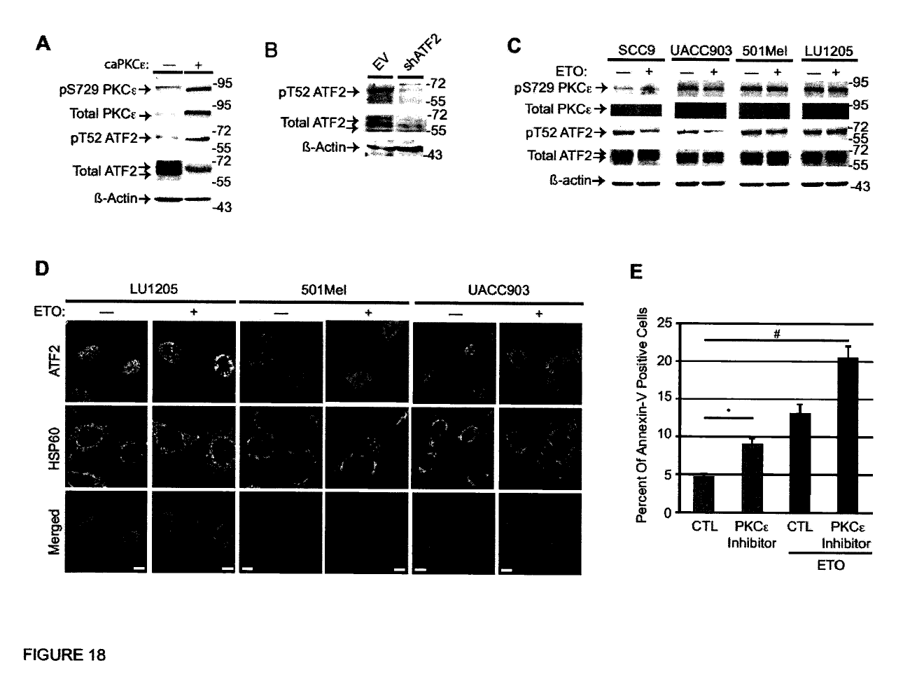

FIG. 19A) is a gel (upper panel) and graph (lower panel) that show primary keratinocytes (NHEK) and melanocytes (HEM), squamous carcinoma (M7, P9, SCC9 ) and melanoma (501Mel, LU1205, WM793 and UACC903) cell lines subject to immunoblot analysis with indicated antibodies. Histogram represents ratio A/B, where A=densitometric ratio values of pT52A/total ATF2 bands, and B=densitometric ratio values of pS729/total PKCε bands. B) is a box plot showing distribution of PKCε in metastatic (left) and primary (right) specimens. PKCε levels are denoted on the Y-axis. While there is a fair degree of overlap in expression between metastatic and primary specimens, the expression overall is significantly higher in the metastatic specimens (P<0.0001, t-statistic—4.257). The mean +/−one standard deviation is depicted by the horizontal bars and is higher in the metastatic specimens. C) is a plot of Kaplan Meier survival curves showing significantly shorter survival in higher PKCε expressers in primary tumors. D) is a diagram showing alignment of amino acid sequences flanking Thr52 in ATF2, as compared to consensus sequence used as PKCε inhibitor peptide. E) is a schematic representation of PKCε-mediated regulation of the subcellular localization, and therefore, the oncogene or tumor suppressor activities of ATF2.

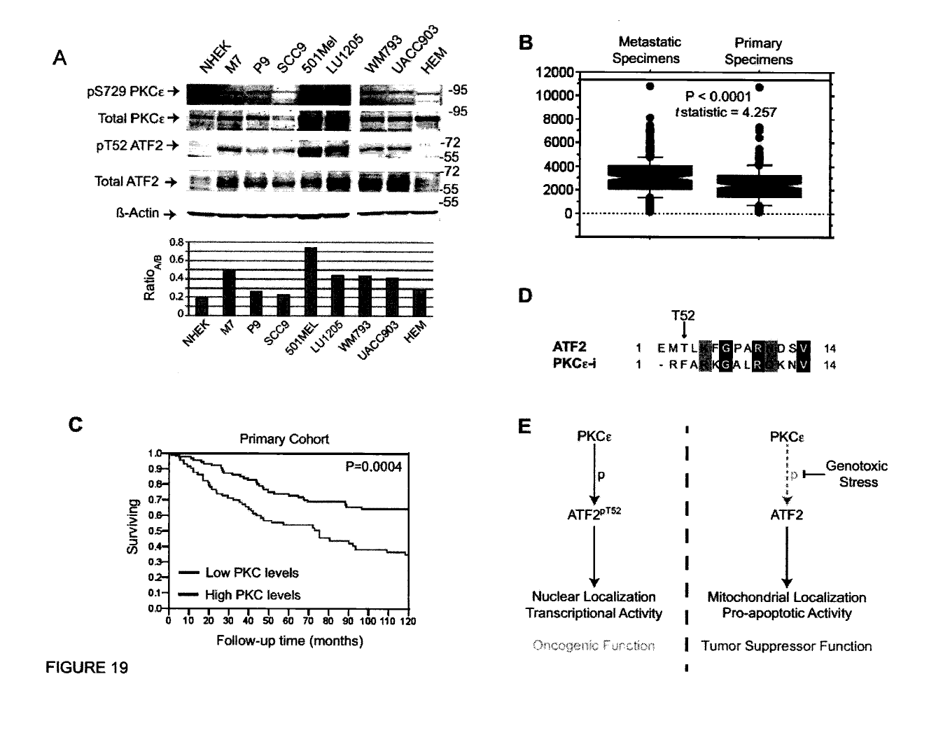

FIG. 20 is a flow-chart that depicts a testing funnel for identification of inducers of ATF2 -mediated apoptotic activity.

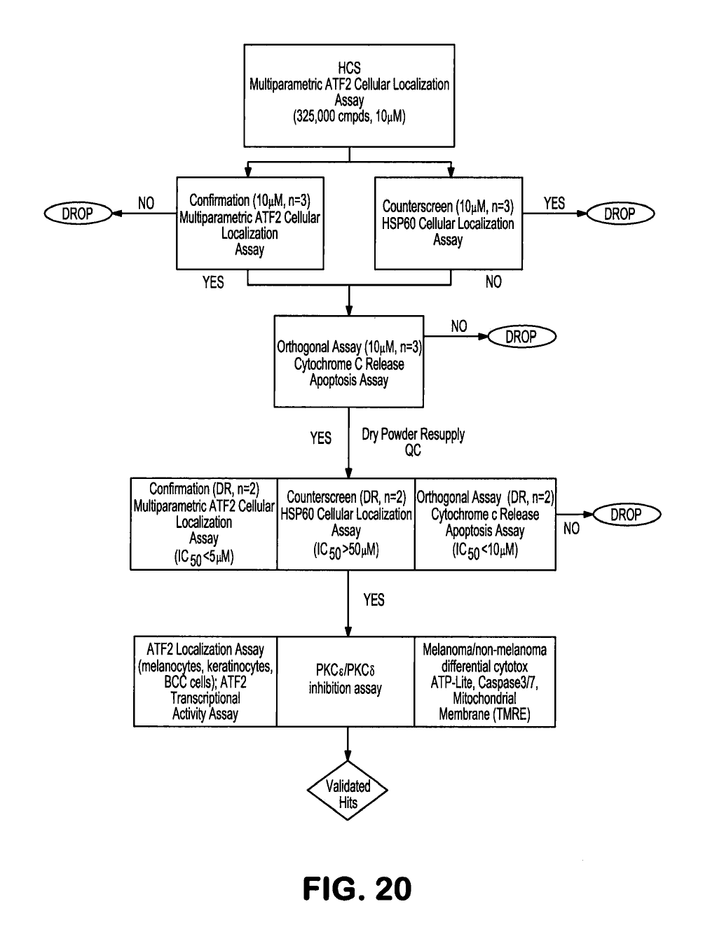

### DETAILED DESCRIPTION OF THE INVENTION

As disclosed herein, ATF2 functions at the mitochondria in response to genotoxic stress. Additionally, ATF2 was found to abrogate formation of high order complexes containing hexokinase-1 (HK1) and voltage-dependent anion-channel-1 (VDAC1) at the outer mitochondrial membrane, disregulating mitochondrial outer membrane permeability and initiating apoptosis. This function is negatively regulated by PKCε phosphorylation of ATF2 on threonine 52, a modification that dictates its nuclear localization. Notably, elevated expression and activity of PKCε was reported in different tumor types, and is associated with poorer outcome and inhibition of tumor cell death. In particular, overexpression of PKCε suffices to promote both the photosensitization of skin and the development of squamous carcinomas. Conversely, the inhibition of PKCε suffices to sensitize tumor cells to cell death, albeit the underlying mechanism for its role in cell death remains largely elusive. It was found that PKCε effect on ATF2 is attenuated in cells subjected to genotoxic stress, enabling nuclear export of ATF2 to the cytosol and its subsequent function at the mitochondria. Such nuclear export and mitochondrial accumulation was not observed in melanoma cells where PKCε activity was constitutively high, thereby blocking mitochondrial ATF2 function. These findings establish the role of PKCε as a key regulator of ATF2 subcellular localization and pro-apoptotic mitochondrial function following genotoxic stress. Notably, ATF2 is among transcription factors shown capable of eliciting oncogenic or tumor suppressor activities, including c-myc, β-catenin, TGFβ and Notch.

While a number of proteins, including the transcription factors Myc, Notch and β-catenin, have been shown capable of exhibiting oncogenic or tumor suppressor activities, the mechanism(s) enabling their opposing function remains largely unknown. Mechanistic insight into the regulation of ATF2 cytosolic or nuclear functions, which are associated with ATF2 tumor suppressor or oncogenic activities, is provided herein, respectively. ATF2 phosphorylation by PKCε determines its nuclear localization and function as a transcription factor. Attenuated ATF2 phosphorylation by PKCε, which occurs in response to genotoxic stress, enables ATF2 nuclear export followed by its localization and function at the mitochondria, where it enhances the permeability of mitochondrial membrane and cell death. Cytosolic localization of ATF2 was observed in non-malignant skin tumors. Genetic studies in mice expressing a transcriptionally inactive form of ATF2 in keratinocytes revealed faster appearance of—and bigger size—skin papillomas, when subjected to DMBA-TPA skin carcinogenesis protocol. Similarly, p53 mutant mice lacking ATF2 in mammary tissues also developed higher incidence of mammary tumors. While these studies are indicative of tumor suppressor activities of ATF2, this transcription factor is shown to exhibit an oncogenic role in certain malignant skin tumors, such as melanoma. Inactivation of ATF2 in melanocytes effectively attenuated the formation of melanoma in the mutant N-Ras/Ink4a model. Similarly, inhibition of ATF2 by short peptides effectively blocked melanoma development. Notably, unlike its cytosolic localization in BCC/SCC, ATF2 was found to be predominantly nuclear in melanoma, which was associated with poor prognosis. Disclosed herein is the mechanistic basis for the predominant nuclear localization of ATF2 given the higher expression of PKCε in the more aggressive melanomas shown here using a large cohort of melanoma tumors. Nuclear localization of ATF2 in melanoma coincides with inactivation of its newly discovered role at the mitochondria, thereby conferring resistance to cell death following genotoxic stimuli. These findings establish that PKCε determines the availability of ATF2 to elicit tumor suppressor, or oncogenic activities.

Mechanistically, ATF2 regulation by PKCε also determines its availability to disrupt HK1/VDAC1 complexes, which reduces mitochondrial membrane integrity in response to genotoxic stress. That perturbation of the HK1:VDAC1 complex confers susceptibility to genotoxic stress-induced apoptosis is consistent with the observation that abrogation of HK1's ability to bind to VDAC1 predisposes cells to cell death. Recent reports highlight the importance of HK1- or HK2- and VDAC1-containing complexes as a crucial early anti-apoptotic component in cellular stress responses, including cancer and pathogenic infection. In cancer, increased levels of the HK:VDAC1 complex are associated with induction of the Warburg effect and a corresponding tumorigenic advantage. Exogenous pathogenic virulence factors, such as Hepatitis E viral Orf3 protein or bacterial FimA, enhance HK1:VDAC interaction to promote host cell survival during infection. In the context of melanoma and skin cancer, it is noteworthy that C-Raf or Akt were reported to modulate HK or VDAC function. While transcription factors such as p53, CREB, Stat3 and Elk-1 reportedly localize at the mitochondria, the finding that ATF2 displaces its transcriptional function with mitochondrial function, thereby swapping its oncogenic activity to a tumor suppressor activity, presents a new paradigm for transcription factor's regulation and function. That the ability of ATF2 to abrogate the VDAC:HK1 complex is perturbed in melanomas offers important insight into the changes that makes these tumors more resistant to apoptosis following exposure to genotoxic stress. Notably, bioinformatics-based analysis of two independent data sets identified high correlation in the expression of ATF2 and VDAC1, which was reduced in the advanced/more malignant tumors.

The discovery of ATF2 regulation by PKCε opens a new avenue of investigation into control of ATF2 transcriptional activities and their possible deregulation in disease states. As phosphorylation of ATF2^{T52} determines its ability to function as a transcription factor, it would be of interest to determine the relationship between T52 and phosphorylation on Thr69/71 by JNK or p38, which has been implicated in its transcriptional activities. The observation that caPKCε overexpression can induce ˜25-fold increase in Jun2-promoter activity, whereas overexpression of ATF2^{T52E} results in only ˜6-fold increase of activity, points to the possibility that PKCε may also augment ATF2 phosphorylation by JNK, p38, or other PKC isoforms. Of interest, high levels of PKCε was associated with upregulation of ERK signaling and melanoma resistance to docetaxel, whereas upregulation of PKC∂ coincided with JNK activation and pro-apoptotic responses. As nuclear import of ATF2 was shown to be affected by its heterodimerization with c-Jun, which is linked to ATF2 phosphorylation by JNK/p38, T52 phosphorylation may also affect ATF2 heterodimerization and degree of transcriptional activities, thereby establishing another layer in the regulation of ATF2 transcriptional activities. Notably, differences in the activity of PKCε among melanomas, and likely other tumor types, will determine if and how much of ATF2 will reach the mitochondria following genotoxic stress, and contribute to genotoxic stress-induced cell death. What determines the high level of PKCε activity in certain tumors, as shown here for melanomas, and what attenuates its effect on ATF2 following genotoxic stimuli, is subject to intense investigation.

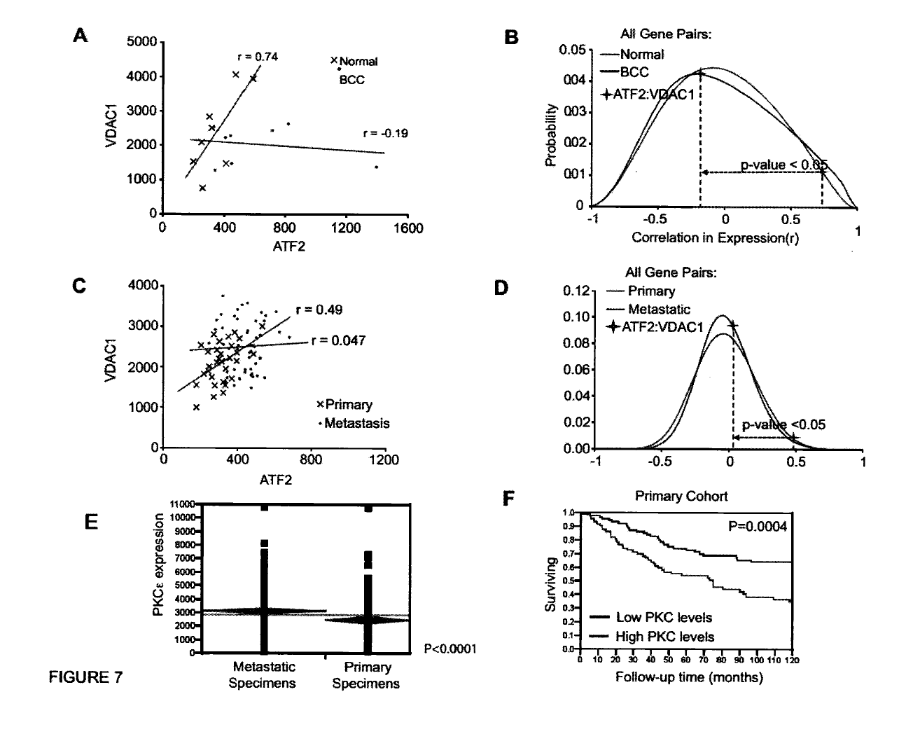

Remarkably, earlier studies identified ATF2-derived peptides of 50 or 10 amino acids that effectively sensitize melanoma cells to apoptosis and inhibit melanoma growth in syngeneic mice, as well as of human melanoma xenografts. Mechanistically, it was demonstrated that these peptides inhibit ATF2 transcriptional activity and increased its cytosolic localization. Disclosed herein is further mechanistic insight into the activity of these peptides, which span the ATF2 sequence from amino acid residues 50-60 or 50-100 and hence contain T52. These peptides likely outcompete PKCε phosphorylation of endogenous ATF2, thereby enabling its cytosolic location and mitochondrial function. Evidence in support of this hypothesis includes the observation that mutation of this peptide within T52 abolished its effect on ATF2 localization and activity. Strikingly, the amino acid sequences flanking Threonine 52 in ATF2 exhibit remarkable similarities with PKC peptide inhibitors and fit the requirements for optimal PKC substrate sequence as determined by an oriented peptide library screen. Further, while most PKC isozymes show strong preference for peptides with basic amino acids at position −2, PKCε also accepts glutamic acid at this position, which is the residue found at the corresponding position in the ATF2 peptide (FIG. 7D).

In a large cohort, it was shown that high PKCε expression in primary melanomas is associated with poor outcome, consistent with ATF2 nuclear localization. The fact that PKCε expression did not retain its independence as a prognostic marker on multivariable analysis suggests that it might not be a primary driving force in tumor metastasis, but rather closely related to another molecular alteration. An alternative explanation is that PKCε expression might be one of drivers of Breslow depth (and tissue invasion), which remained an independent prognostic factor in the multivariable model. This notion is consistent with findings that ATF2^{T52E} does not appear to overtly confer transforming abilities, such as anchorage independent growth in soft agar (data not shown) or proliferative advantage, but does confer survival advantage in regards to genotoxic stress. In such regard to genotoxic stress response, it is possible that phosphorylation of ATF2 on T52 may also impact its role in DNA damage response by affecting its phosphoregulation on S490/498 by ATM. However, it is plausible that phosphorylation of ATF2 on T52 would enhance or alter its transcriptional output. Those could include ATF2 control of pro-proliferative/survival proteins, increased transcriptional activities of its heterodimeric partner c-Jun, with concomitant effect on RACK1 and PDK1 signaling. Notably, PKCε is upregulated in melanomas that tend to metastasize, providing a mechanism antagonizing ATF2 mitochondrial function and revealing why nuclear localization of ATF2 is associated with poor prognosis. Correlation of elevated expression and activity of PKCε with poor prognosis and resistance to chemotherapy has been reported for numerous cancers. It would be beneficial to determine the regulation and status of ATF2 in these tumors. Notably, efforts to target PKCε as cancer therapy met limited success, primarily because of the similarity with PKCδ, whose function opposes that of PKCε. As disclosed herein, there is evidence suggestive that compounds that would induce cytosolic localization of ATF2 in melanoma cells may represent a new class of PKCε inhibitors.

The role of ATF2 at the mitochondria, following genotoxic stress has been established, thereby revealing its contribution to stress-induced cell death. The degree of PKCε phosphorylation determines ATF2 ability to function at the mitochondria, where its contribution to cell death are consistent with its tumor suppressor functions associated with cytoplasmic localization. Constitutively high expression/activity of PKCε, as seen in melanomas, attenuates ATF2 mitochondrial function and enhances its transcriptional activity, consistent with its nuclear localization, which has been associated with its oncogenic activity. It is the subcellular localization of ATF2, regulated by PKCε, which dictates the nature of its ability to elicit oncogenic or tumor suppressor activities.

The transcription factor ATF2 elicits oncogenic activities in melanoma, and tumor suppressor activities in non-malignant skin cancer. The ATF2 tumor suppressor function is determined by its ability to localize at the mitochondria, where it alters membrane permeability following genotoxic stress. The ability of ATF2 to reach the mitochondria is determined by PKCε, which directs ATF2 nuclear localization. Genotoxic stress attenuates PKCε effect on ATF2, enables ATF2 nuclear export and localization at the mitochondria, where it perturbs the HK1-VDAC1 complex, increases mitochondrial permeability and promotes apoptosis. Significantly, high levels of PKCε, as seen in melanoma cells, block ATF2 nuclear export and function at the mitochondria, thereby attenuating apoptosis following exposure to genotoxic stress. In melanoma tumor samples, high PKCε levels associates with poor prognosis. These findings provide a framework for understanding how subcellular localization enables ATF2 oncogenic or tumor suppressor functions.

In the course of elucidating mechanisms underlying the opposing activities of ATF2, it was discovered that the tumor suppressor function of ATF2 requires its cytosolic localization. Tissue microarrays (TMAs) of basal cell carcinomas (BCC) or squamous cell carcinomas (SCC) revealed that ATF2 is cytosolic, whereas in melanoma tumors ATF2 is primarily nuclear, consistent with constitutive transcriptional activation of its targets. Notably, in melanoma patients, nuclear ATF2 is associated with poor prognosis, pointing to the use of ATF2 localization as a marker of disease outcome.

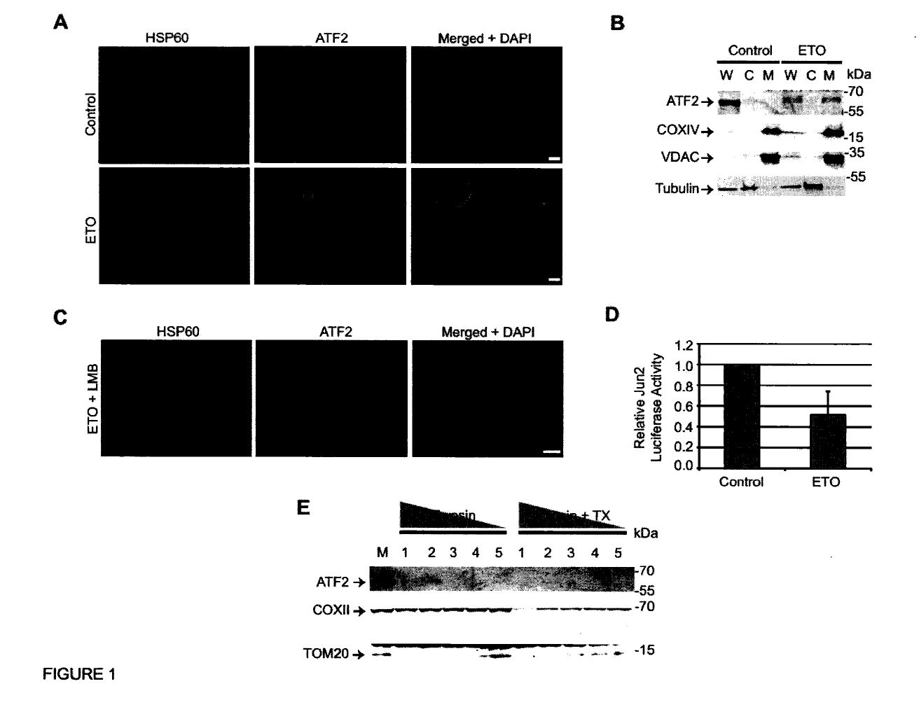

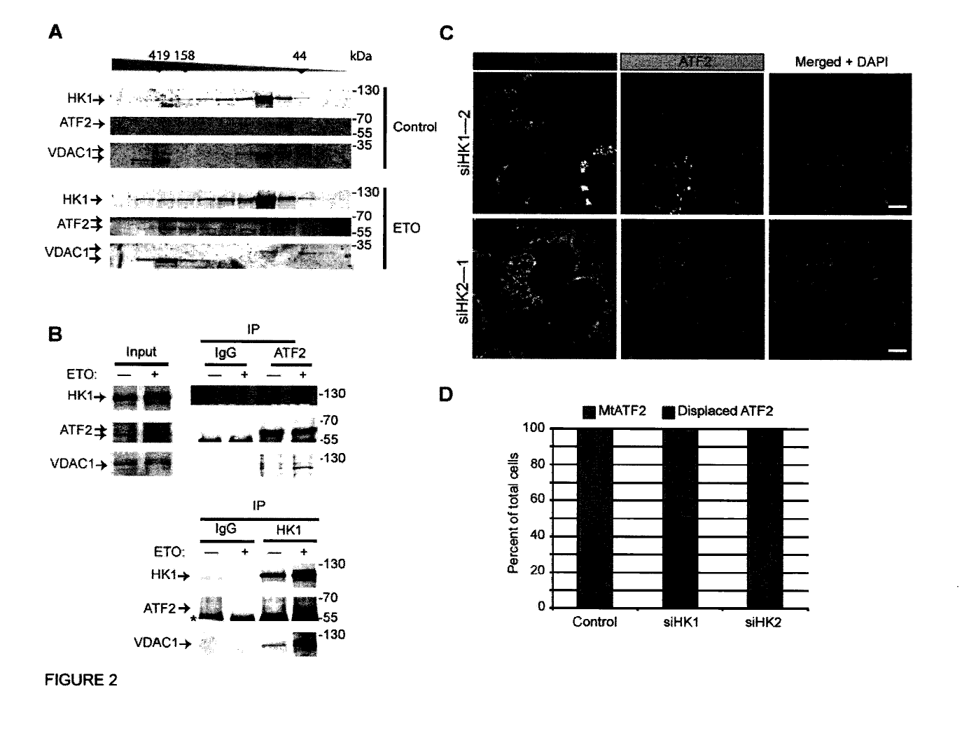

In evaluating the possible cytosolic function of ATF2 in SCC/BCC, ATF2 was found to associate with the mitochondrial outer membrane proteins hexokinase 1 and VDAC1. ATF2 mobilization to the mitochondria is seen in various cells, including normal melanocytes, keratinocytes, fibroblasts and BCC/SCC cells, as well as in early stage melanomas, but not in advanced melanoma (FIG. 1A, 1C). ATF2 localization at the outer membrane of the mitochondria following genotoxic stress enables its assembly into a complex with VDAC1 and HK1 (FIG. 1B). Prior to ATF2 translocation, HK1 and VDAC complex maintains mitochondrial membrane homeostasis and is implicated in mitochondrial biogenesis. Once bound to HK1 and VDAC, ATF2 interferes with their ability to maintain mitochondrial membrane integrity, thereby increasing permeability and promoting cell death. That this does not occur in more advanced metastatic melanoma highlights a novel mechanism by which melanoma may acquire chemoresistance. Furthermore, studies have also identified the mechanism underlying ATF2's subcellular localization and its availability to mitochondria. It was found that PKCε-mediates phosphorylation of residue Thr52 results in ATF2 nuclear localization (FIG. 2A), and thereby governs its oncogenic or tumor suppressor activities. Constitutively high levels of PKCε melanoma, prevent ATF2's nuclear export and its mitochondrial tumor suppressor function. Analysis of >500 melanoma TMAs confirmed that PKCε expression is associated with poor prognosis (FIG. 2E), consistent with earlier findings pointing to an association of melanoma with nuclear ATF2. Inhibition of PKCε activity in MeWO melanoma cells that are resistant to genotoxic stress induces apoptosis, which further increases when administered in the presence of genotoxic stress (FIG. 1D). Notably, both 10 and 50 amino acid long peptides derived from ATF2 (amino acids 50-60 or 50-100, respectively) sensitized melanoma cells (but not melanocytes) to apoptosis. These peptides effectively caused nuclear exclusion of ATF2. Furthermore, mutation of the peptide at ATF2 amino acid 52 abolished its ability to elicit these effects, consistent with the identification of this residue as a PKCε phosphorylation site and its role in nuclear localization of ATF2. Thus, facilitating ATF2 nuclear export and restoring a cytosolic function of the transcription factor ATF2 in melanoma cells, a strategy that sensitizes these cells to apoptosis following exposure to genotoxic stimuli in melanoma, provides a novel therapeutic modality that could overcome existing treatment barriers.

The invention includes antibodies immunoreactive with or which bind to ATF2, for example. Antibody which consists essentially of pooled monoclonal antibodies with different epitopic specificities, as well as distinct monoclonal antibody preparations are provided. Monoclonal antibodies are made from antigen containing fragments of the protein by methods well known to those skilled in the art (Kohler, et al., Nature, 256:495, 1975). The term antibody as used in this invention is meant to include intact molecules as well as fragments thereof, such as Fab and F(ab′)₂, which are capable of binding an epitopic determinant on the desired antigen/protein. The antibodies of the invention include antibodies which bind to ATF2, and particularly ones that specifically bind to the T52 residue when phosphorylated.

The term “antibody” as used in this invention includes intact molecules as well as fragments thereof, such as Fab, F(ab′)₂, and Fv which are capable of binding the epitopic determinant. These antibody fragments retain some ability to selectively bind with its antigen or receptor and are defined as follows:

(1) Fab, the fragment which contains a monovalent antigen-binding fragment of an antibody molecule can be produced by digestion of whole antibody with the enzyme papain to yield an intact light chain and a portion of one heavy chain;

(2) Fab′, the fragment of an antibody molecule can be obtained by treating whole antibody with pepsin, followed by reduction, to yield an intact light chain and a portion of the heavy chain; two Fab′ fragments are obtained per antibody molecule;

(3) (Fab′)₂, the fragment of the antibody that can be obtained by treating whole antibody with the enzyme pepsin without subsequent reduction; F(ab′)₂ is a dimer of two Fab′ fragments held together by two disulfide bonds;

(4) Fv, defined as a genetically engineered fragment containing the variable genetically fused single chain molecule.

Methods of making these fragments are known in the art. (See for example, Harlow and Lane, Antibodies: A Laboratory Manual, Cold Spring Harbor Laboratory, New York (1988), incorporated herein by reference).

As used in this invention, the term “epitope” means any antigenic determinant on an antigen to which the paratope of an antibody binds. Epitopic determinants usually consist of chemically active surface groupings of molecules such as amino acids or sugar side chains and usually have specific three dimensional structural characteristics, as well as specific charge characteristics.

Antibodies which bind to phosphorylated ATF2 polypeptide of the invention can be prepared using an intact polypeptide or fragments containing small peptides of interest as the immunizing antigen. The polypeptide used to immunize an animal can be derived from translated cDNA or chemical synthesis which can be conjugated to a carrier protein, if desired. Such commonly used carriers which are chemically coupled to the peptide include keyhole limpet hemocyanin (KLH), thyroglobulin, bovine serum albumin (BSA), and tetanus toxoid. The coupled peptide is then used to immunize the animal (e.g., a mouse, a rat, or a rabbit).

If desired, polyclonal or monoclonal antibodies can be further purified, for example, by binding to and elution from a matrix to which the polypeptide or a peptide to which the antibodies were raised is bound. Those of skill in the art will know of various techniques common in the immunology arts for purification and/or concentration of polyclonal antibodies, as well as monoclonal antibodies (see for example, Coligan, et al., Unit 9, Current Protocols in Immunology, Wiley Interscience, (1991), incorporated herein by reference).

The following examples are intended to illustrate but not limit the invention.

### EXAMPLE 1

### Genotoxic Stress Induces ATF2 Nuclear Export and Mitochondrial Localization

This example demonstrates that ATF2 localizes at the mitochondrial outer membrane in response to genotoxic stress.

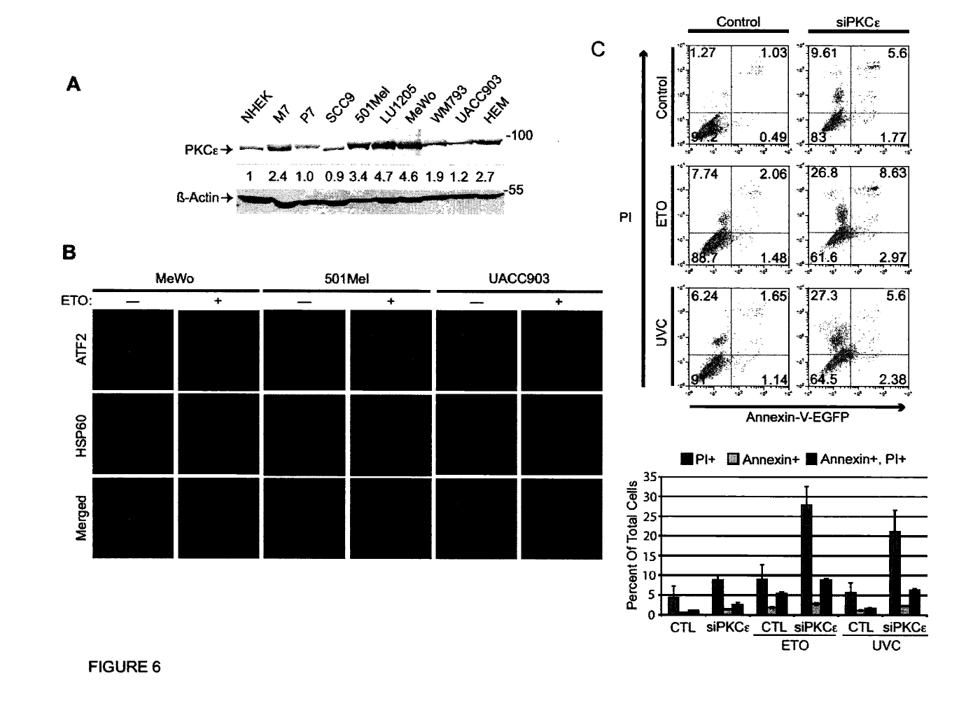

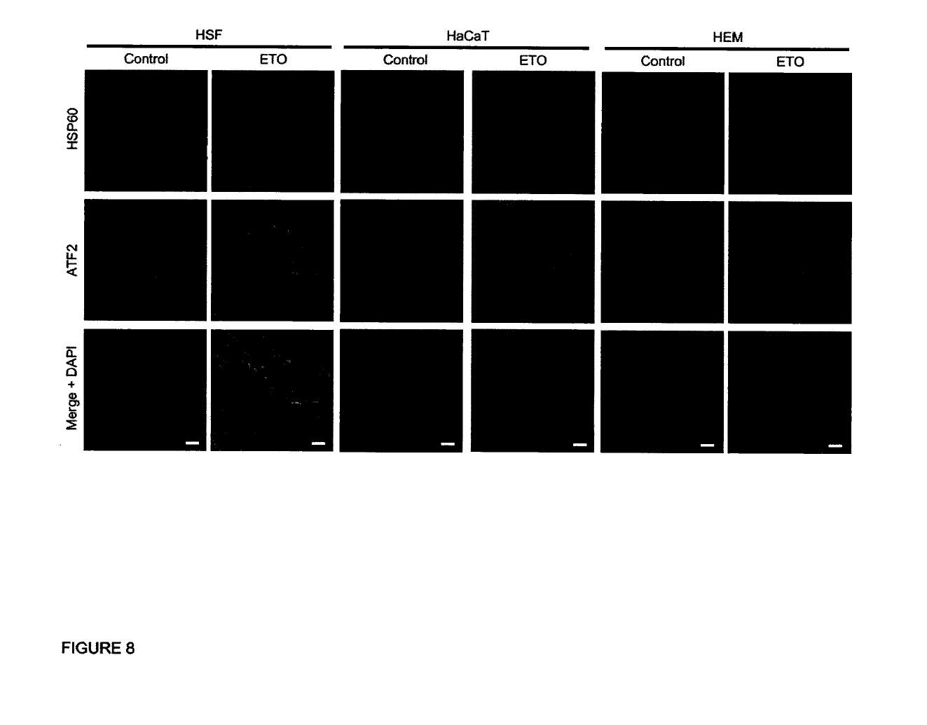

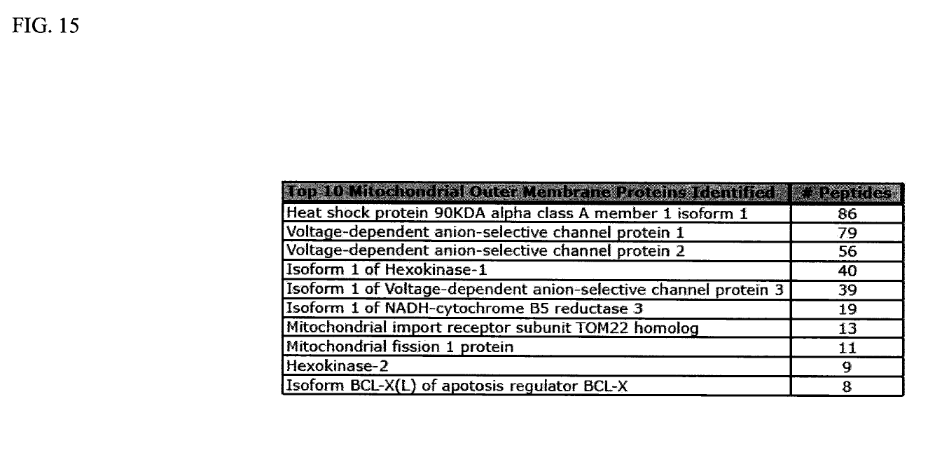

While ATF2 cytoplasmic localization has been previously reported in various tissues, the function of cytoplasmic ATF2 has not yet been described. Based on the observed cytosolic localization of ATF2 in squamous cell carcinoma (SCC) tumors, an SCC line (SCC9 ) was employed to analyze potential ATF2 cytoplasmic function. Mass spectrometric analysis of cytosol-localized ATF2-bound proteins in SCC9 cells identified a cluster of mitochondria-outer membrane related proteins, suggestive of an ATF2 mitochondrial interaction (FIG. 15). Indeed, while SCC9 cells grown under non-stressed conditions exhibit predominantly nuclear localization of ATF2 , genotoxic stress induced by etoposide resulted in its accumulation at the mitochondria in ˜64% of the cells subjected to this treatment (FIG. 1A). Similarly, other genotoxic insults, including ultraviolet C (UVC, 20J/m²) and ionizing irradiation (IR, 5Gy) also prompted the mitochondrial localization of ATF2 (FIG. 1A). The localization of ATF2 at the mitochondria following genotoxic stimuli was also seen in other cell types, including normal human fibroblasts (HSF, FIG. 8), primary human keratinocytes (NHEK, FIG. 8) and melanocytes (HEM, FIG. 8), as well as in other squamous cell carcinoma cell lines (M7 and P9; FIG. 8). Whereas some melanoma cell lines (UACC903) exhibit partial enrichment of ATF2 at the mitochondria (FIGS. 6D & 8), a number of melanoma cell lines (LU1205 and 501Mel, FIGS. 6 & 8) no longer exhibited ATF2 export or localization at the mitochondria following genotoxic stimuli, with the exception of IR (FIG. 8), suggesting that malignant melanomas are largely resistant to genotoxic stress-induced mitochondrial translocation of ATF2. The extent of ATF2 mitochondrial localization differs among the different cultures and type of stimuli, indicative of variation in the cellular response and control of such translocation in each of these cultures. Biochemical fractionation of SCC9 cells confirmed that exposure to genotoxic stress results in the accumulation of ATF2 in purified mitochondrial fractions, as indicated by mitochondrial markers COXI, COXIV and VDAC1 (FIG. 1B).

Pre-treatment of SCC9 cells with the nuclear export inhibitor, leptomycin B (LMB), prevented mitochondrial accumulation of ATF2 following etoposide treatment, indicating that ATF2 is exported from the nucleus after genotoxic stress (FIG. 1A, 5^{th} row). Consistent with this finding, a ˜50% reduction of luciferase activity driven by the Jun2 promoter was observed, which is targeted by ATF2 (FIG. 1C). These findings suggest that genotoxic stress induces nuclear export and mitochondrial localization of ATF2, thereby attenuating its transcriptional activity.

ATF2 protein does not harbor a canonical mitochondrial localization or peptide-processing signal, which could facilitate import or insertion into mitochondrial membranes, suggesting that ATF2 mitochondrial localization requires interaction with a mitochondrial protein(s). In order to investigate this possibility, the location of ATF2 accumulation at the mitochondria was determined. Mitochondria possess inner and outer membranes, thus exhibiting four possible locations for protein/protein interaction: the surface of the mitochondrial outer membrane (MOM), the intermembrane space (IMS), the mitochondrial inner membrane (MIM), or the mitochondrial inner matrix. ATF2 localization at the MOM versus transport into the mitochondria was evaluated in order to determine where ATF2 resides. Limited proteolysis protection assays with enriched mitochondrial fractions obtained from etoposide-treated SCC9 cells revealed that ATF2 was readily degraded by protease in the absence of detergent, consistent with the degradation pattern of the MOM component, TOM20, but differing from the pattern seen with inner matrix protein, COXII, which is degraded only in the presence of detergent (FIG. 1D). These results suggest that in response to genotoxic stress, ATF2 localizes at the MOM.

### EXAMPLE 2

### ATF2 Interacts with HK1:VDAC1 Complexes Following Genotoxic Stress

This example illustrates that ATF2 interacts with VDAC1 and HK1 complex at the mitochondria.

To determine ATF2 function at mitochondria, interaction of various MOM protein(s) with ATF2 was examined, with a focus on proteins identified in a mass spectrometry analysis (FIG. 15). Among the top-ranking candidates was a group of proteins known to form MOM complexes following exposure to genotoxic stress, including Hexokinase-1 (HK1), and Voltage-Dependent Anion Channel 1 (VDAC1) (FIG. 8).

In response to various forms of stress, HK1 and HK2 bind to VDAC1, which oligomerizes to form large molecular weight complexes. To determine whether ATF2 is part of these complexes, an assessment was made of the distribution of ATF2, HK1 and VDAC1 in fractions obtained following fast protein liquid chromatographic (FPLC) analysis using a gel filtration column. Whereas prior to stress, ATF2 did not co-distribute to fractions with VDAC1 and HK1, after etoposide treatment, all three proteins were found within high molecular weight fractions (FIG. 2A).

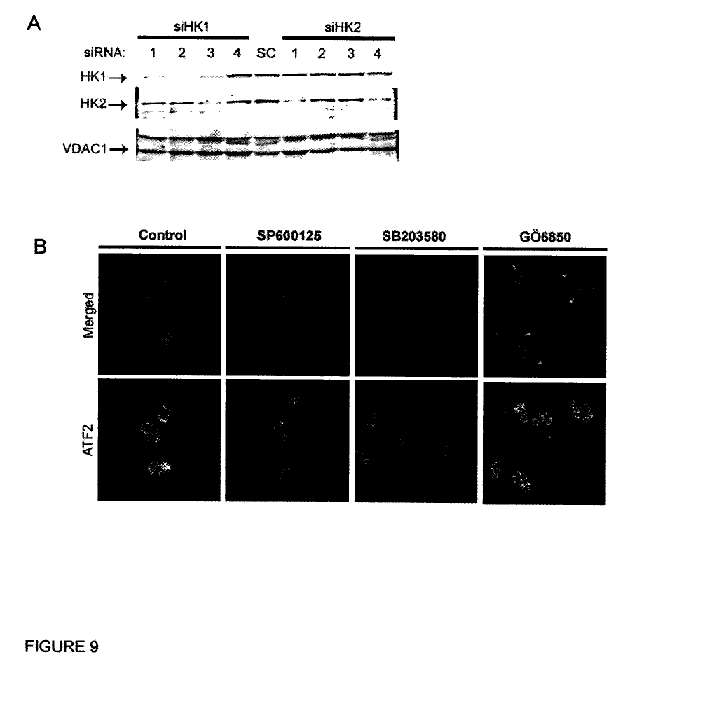

To confirm the ATF2/VDAC1 interaction, as well as interaction with HK1, endogenous ATF2 or HK1 from cell lysates of SCC9 cells prior to and after etoposide treatment were immunoprecipitated. ATF2 interaction with HK1 and with high molecular weight (˜130 kDa) tetrameric VDAC1 oligomers, detectable with the aid of crosslinker, was clearly seen after etoposide treatment (FIG. 2B, upper and lower). Interestingly, knockdown of HK1, but not of HK2, abrogated mitochondrial localization of ATF2 following etoposide treatment, indicating that ATF2 requires HK1 to localize to mitochondria after genotoxic stress (FIGS. 2C, 2D and 9A). Coupled with the gel filtration data, these observations suggest that ATF2 interacts with both HK1 and VDAC1 after genotoxic stress.

### EXAMPLE 3

### PKCε-Phosphorylation Negatively Regulates ATF2 Mitochondrial Localization

This example illustrates that PKCε phosphorylation of ATF2 on Thr52 negatively regulates its mitochondrial localization.

Phosphorylation of ATF2 by p38 or JNK on Thr69/71 is required for its dimerization with members of the AP-1 transcription factor family and for transcriptional activity. However, phosphorylation of Thr69/71 was not required for ATF2 mitochondrial localization, as inhibition of either p38 or JNK activity, either by specific pharmacological inhibitors did not alter its nuclear localization (FIG. 9B).

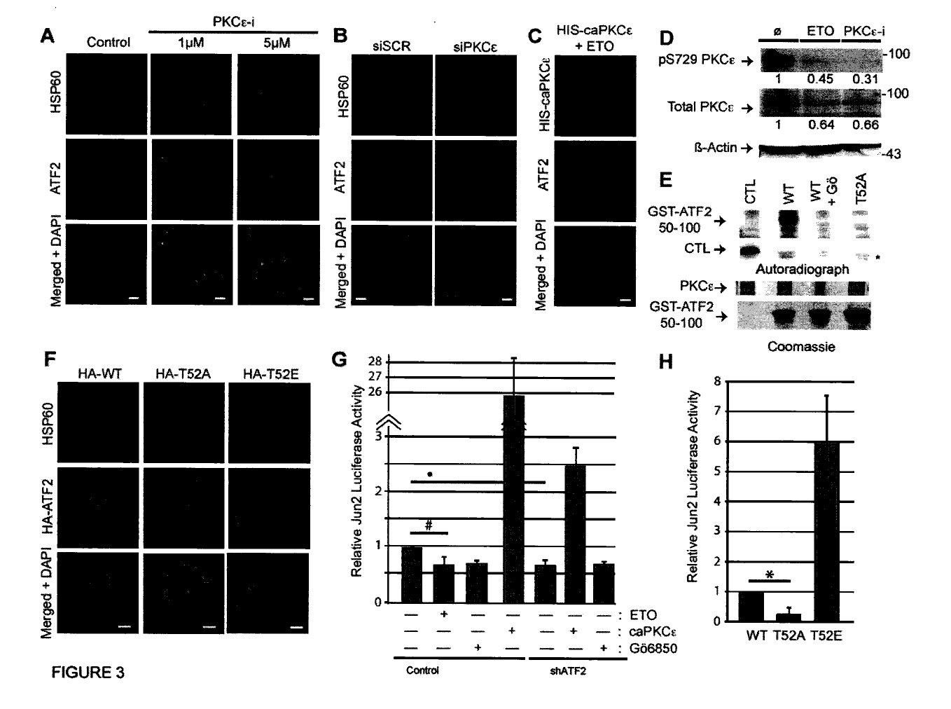

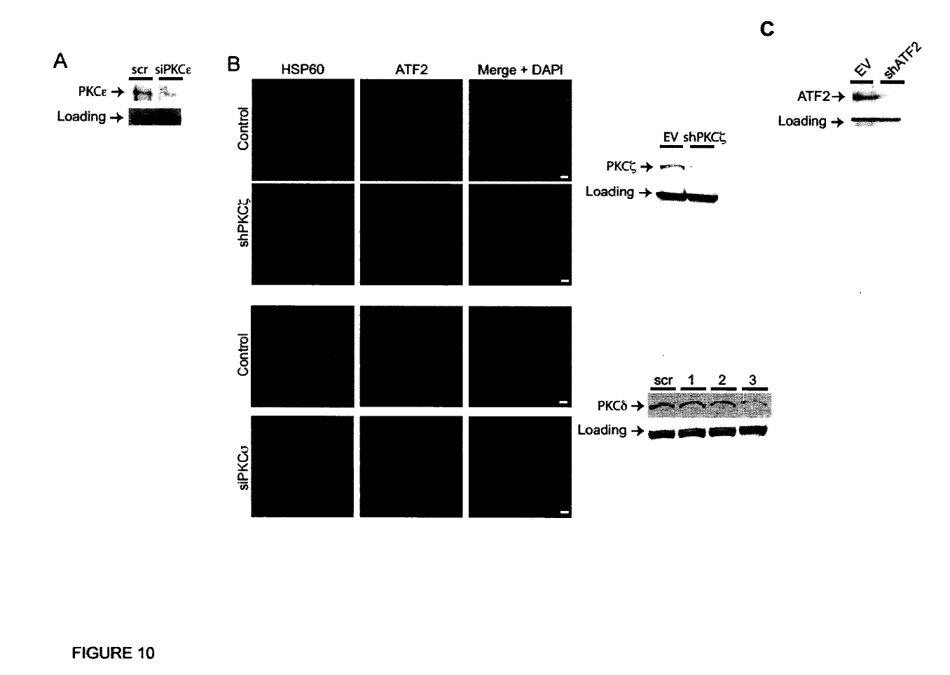

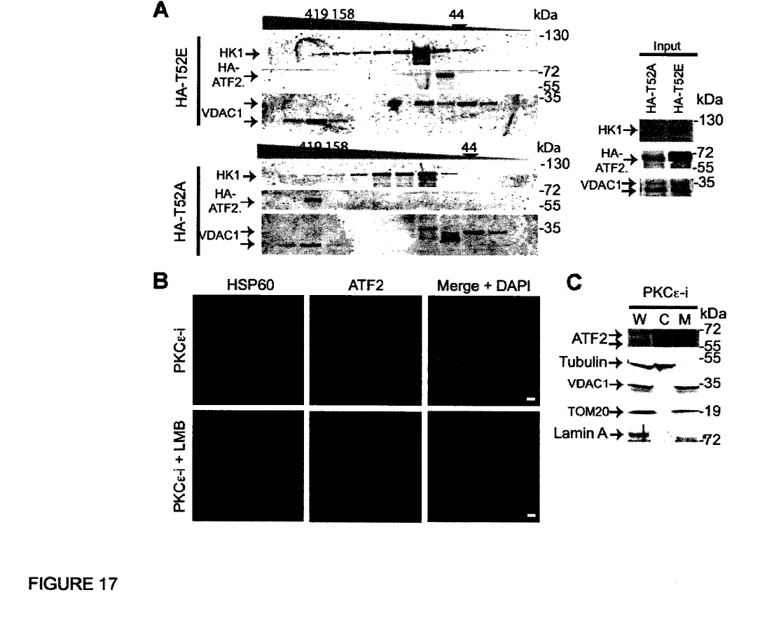

To identify kinases that might affect ATF2 mitochondrial localization, the ATF2 protein sequence was subjected to phosphorylation site scanning (http://scansite.mit.edu). This analysis predicted an uncharacterized, but highly probable PKCε/∂/ζ phosphorylation site at ATF2 residue Thr52. To evaluate the possible role of PKCε in the mitochondrial function of ATF2, immunofluorescent staining analysis was employed to assess possible changes in ATF2 mitochondrial localization following PKCε inhibition. Strikingly, treatment with the PKCε translocation inhibitor (PKCε-i) promoted mitochondrial localization of ATF2, even in the absence of etoposide, as detected by both immunofluorescent staining and mitochondrial fractionation (FIG. 3A, 17F). Consistent with these findings, PKCε knockdown (FIG. 10A) with corresponding siRNA promoted mitochondrial localization of ATF2 (FIG. 3B), while overexpression of a constitutively active form of PKCε (caPKCε), which is mutated in its pseudosubstrate auto-inhibitory domain, abolished ATF2 accumulation at mitochondria following etoposide treatment (FIG. 3C). As with the genotoxic stress-induced nuclear export of ATF2 (FIG. 1A), its mitochondrial translocation following inhibition of PKCε was efficiently blocked by pre-treatment by leptomycin B (FIG. 17E). To determine whether atypical PKC forms, such as ∂ or ζ, alter ATF2 localization each isoform was individually knocked down, but only a negligible effect on mitochondrial ATF2 localization was observed (FIG. 10B). These observations indicate a specific role of PKCε in negative regulation of ATF2 mitochondrial localization. Accordingly, it is reasonable to expect that genotoxic stress would attenuate PKCε effect on ATF2, enabling its mitochondrial localization. Indeed, PKCε phosphorylation on Ser729, an indicator of active PKCε, decreased following overnight treatment with etoposide or with PKCε-i (FIG. 3D). These data suggest overall that PKCε effect on nuclear localization of ATF2 is attenuated following genotoxic stress, enabling ATF2 nuclear export and mitochondrial localization.

To determine whether Thr52, the predicted PKCε site for phosphorylation of ATF2, regulates mitochondrial translocation of ATF2, phosphorylation of ATF2 by PKCε in vitro was assessed, as well as the localization of ATF2^{T52A} (a non-phosphorylatable alanine phosphomutant) and ATF2^{T52E} (a glutamic acid phosphomimic). In vitro kinase assays employing either a GST-tagged ATF2 50-100 amino acid peptide with T52 wild-type (WT) or mutant (T52A) as substrates revealed that PKCε phosphorylates WT but not the ATF2^{T52A} peptide. This phosphorylation is inhibited upon addition of PKCε catalytic inhibitor, Gö6850 (FIG. 3E). Together, these data indicate that Thr52 is a PKCε phosphoacceptor site. Consistent with nuclear localization observed in the presence of caPKCε, the phosphomimic ATF2^{T52E} mutant exhibited constitutive nuclear localization (FIG. 3F), whereas the non-phosphorylatable ATF2^{T52A} mutant localized to the cytoplasm and mitochondria, even in the absence of genotoxic stress (FIG. 3F). Gel filtration analysis revealed that, ATF2^{T52A}, but not ATF2^{T52E}, distributed to large molecular weight fractions containing VDAC1 (FIG. 17D). Interestingly, expression of ATF2^{T52A} revealed less HK1 within the high molecular weight VDAC1-containing fractions, pointing to the possibility that ATF2^{T52A} may attenuate the interaction between HK1 and VDAC1. Collectively, these findings establish that ATF2 mitochondrial localization is regulated by PKCε through phosphorylation at T52.

Given the effect of PKCε on ATF2 subcellular localization its effect on ATF2 transcriptional activity was next assessed. Strikingly, overexpression of constitutively active PKCε (caPKCεalone led to a marked increase in Jun2-luciferase activity, indicative of ATF2 transcriptional activity, which was largely attenuated upon knockdown of ATF2 (FIG. 3G). Interestingly, PKCε inhibition with Gö6850 or suppression by treatment with ETO appeared to reduce Jun2-luciferase activity to similar extents as knockdown of ATF2, suggesting that PKCε also contributes to the basal transcriptional activity of ATF2 in these cells. Consistently, reconstitution of ATF2 stable-knockdown SCC9 cells (employing shRNA targeting ATF2 3′-UTR, FIG. 10C) with ATF2^{T52E} also increased Jun2-luciferase activity compared to wild-type ATF2. Conversely, mutation of T52 to alanine abolished ATF2 transcriptional activity (FIG. 3H). These findings indicate that by regulating ATF2 nuclear localization, PKCε not only determines ability of ATF2 to reach the mitochondria, but also its ability to elicit its transcriptional activity.

### EXAMPLE 4

### Mitochondria-Localized ATF2 Compromises Outer-Membrane Integrity by Disrupting HK1:VDAC1 Complexes

This example illustrates that ATF2^{T52A} displaces HK1 from mitochondria and abrogates HK1:VDAC association, promoting mitochondrial leakage, cytochrome C release and cell death.

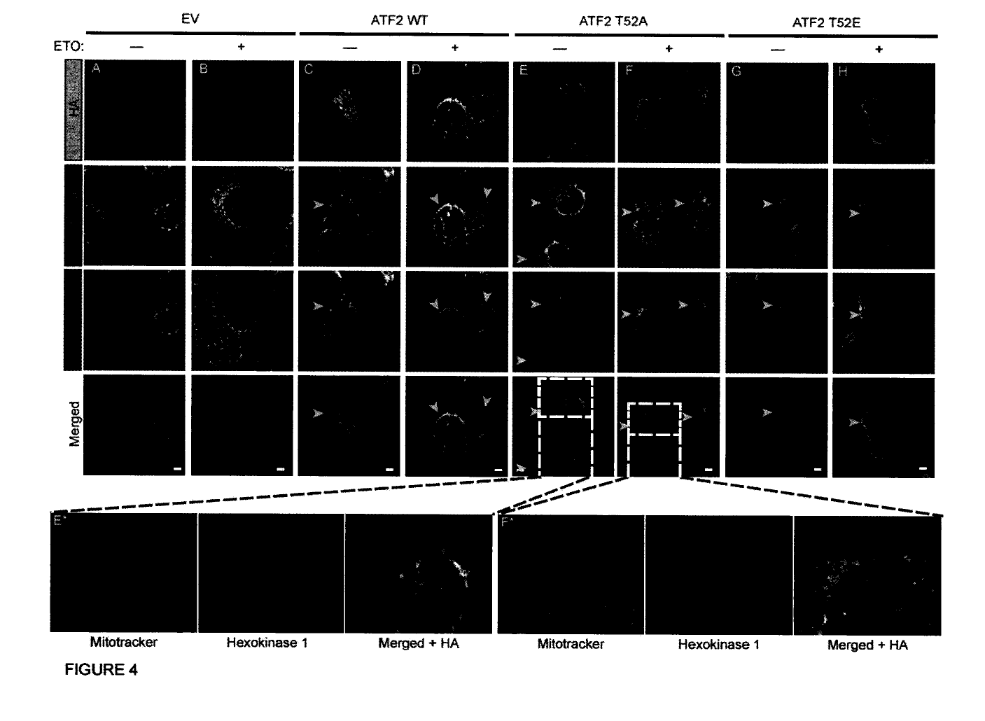

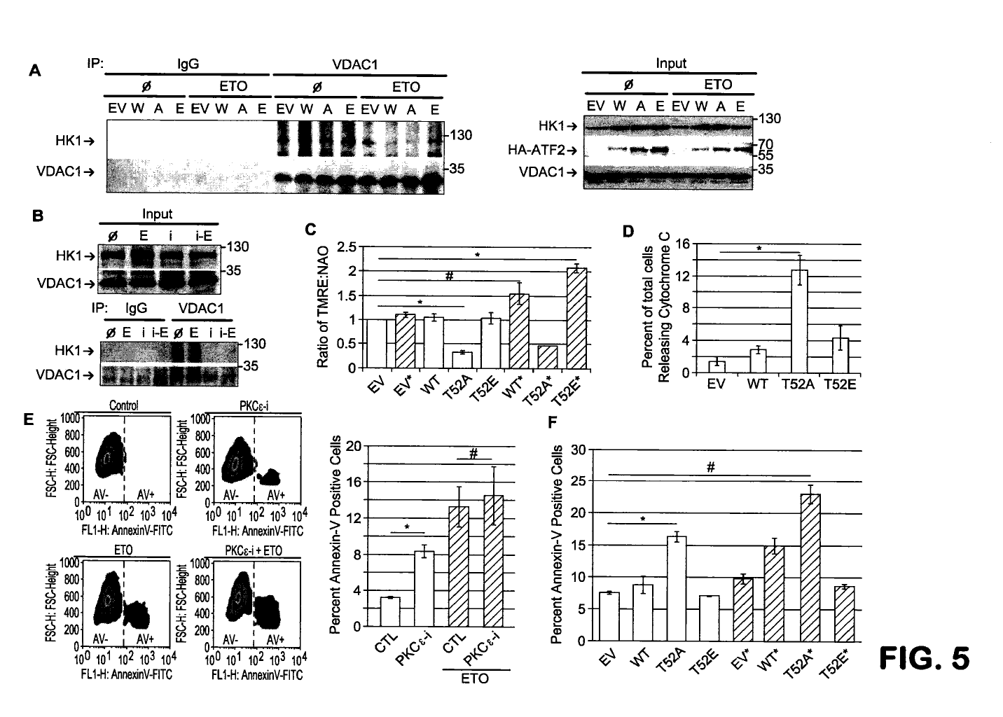

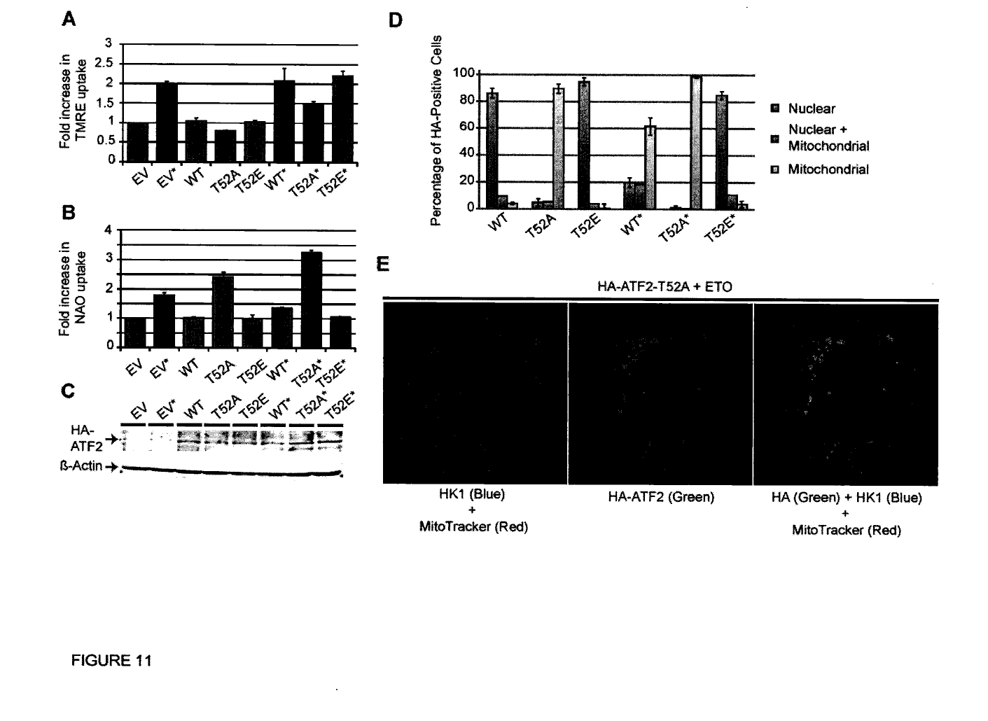

VDAC1 functions as a channel facilitating passage of small molecules and ions bi-directionally through the MOM. Stress-induced mitochondrial leakage is directly enhanced by VDAC1, as VDAC1 overexpression is sufficient to induce membrane leakage and cell death. Disruption of VDAC interaction with HK1 or HK2 sensitizes cells to stress, compromising membrane potential and promoting leakage of molecules from the IMS. Conversely, HK1 or HK2 binding to the MOM promotes resistance to stress and increased cell survival. In order to determine whether the HK1/VDAC1 complex was perturbed by interaction with ATF2, HK1 accumulation at mitochondria following ectopic expression of ATF2 phosphomutants that preferentially localize to either the nucleus (ATF2^{T52E}) or mitochondria (ATF2^{T52A}) (FIGS. 4, 11D) was evaluated. Under basal conditions, HK1 is localized to the cytoplasm and mitochondria (FIG. 4A). Notably, HK1 co-localization with mitochondria was enhanced after etoposide treatment (FIG. 4B). In cells transfected with ATF2^{T52E}, HK1 accumulation at mitochondria was similar to that seen in control cells prior and after etoposide treatment (FIG. 4G, H). However, ATF2^{T52A} overexpression abrogated HK1 accumulation at the mitochondria prior to and following genotoxic stress, as indicated by decreased distinct co-localization of HK1 and MitoTracker (FIG. 4E, F and insets 4E*, F*). Notably, cells expressing ATF^{T52A} and treated with etoposide exhibited more fragmented mitochondria compared to cells expressing empty vector, WT ATF2 or ATF2^{T52E} (Figure insets 4E* and F*, compared to 4 A, B, C, G and H). Further, the expression ATF2^{T52A} promoted the appearance of punctuated, HK1-positively staining structures that co-localized near, but not along the MitoTracker-labeled mitochondrial structures (FIG. 4E, F and insets E* and F*, and 11E). These results indicate that ATF2 mitochondrial translocation perturbs HK1/VDAC1 complexes and MOM integrity. Overexpression of WT ATF2 was also sufficient to abrogate HK1 localization during genotoxic stress, as during such conditions WT ATF2 was able to translocate to the mitochondria (FIG. 4D). Although ATF2 may also affect VDAC1 multimerization, an event that promotes mitochondrial leakage and cell death, changes in VDAC1 multimerization were not observed, as assessed by crosslinking and non-reducing immunoblot analysis, in the presence of either WT or the T52 ATF2 mutants (data not shown). The effect of ATF2 on HK1 association with VDAC1 in the presence or absence of etoposide was next evaluated. Consistent with immunostaining data, overexpression of ATF2^{T52A} abrogated HK1 association with VDAC that was immunprecipitated under basal conditions and following genotoxic stress. Overexpression of wild-type ATF2 abrogated HK1-VDAC1 complex only after etoposide treatment whereas the ATF2^{T52E} did not have a marked effect on HK1 binding to VDAC1 (FIG. 5A). In agreement, HK1 association with VDAC1 was also decreased following treatment of SCC9 cells with PKCε inhibitor alone or PKCε inhibitor in the presence of etoposide (FIG. 5B). These data indicate that mitochondria-localized ATF2 attenuates HK1 ability to form a complex with VDAC.

### EXAMPLE 5

### ATF2 Localization at the Mitochondria Reduces Mitochondrial Potential and Sensitizes Cells to Genotoxic Stress-Induced Death

This example illustrates the effects of changes in mitochondrial dynamics and subcellular localization of ATF2 WT, T52A and T52E

To assess possible changes in mitochondrial function following genotoxic stress, mitochondrial membrane potential changes indicative of leakage were monitored. Alterations in mitochondrial membrane potential can be measured by short incubation of cells with tetramethylrhodamine ethyl ester (TMRE), a cationic dye preferentially taken up by mitochondria. Fluorescence-activated cell sorting (FACS) was subsequently employed to quantify TMRE uptake as an indicator of relative mitochondrial membrane potential. Consistent with previous reports, low-dosage etoposide induced an initial increase in the amount of TMRE taken up by control SCC9 cells (FIG. 11A), whereas treatment with increased doses of etoposide reduced TMRE uptake (data not shown). Cytotoxicity and mitochondrial leakage seen following treatment with high doses of etoposide have also been reported.

SCC9 cells transfected with ATF2^{T52E} did not exhibit alterations in TMRE uptake compared to cells transfected with empty vector or ATF2^{WT}. These, as well as control transfected cells, showed increased TMRE uptake following exposure to genotoxic stress (FIG. 11A). In contrast, cells transfected with ATF2^{T52A} exhibited decreased TMRE uptake (FIG. 11, 11C). These results are consistent with immunostaining data indicating an antagonizing function of mitochondrial ATF2 in formation of HK1/VDAC1 complexes, and consequently in MOM integrity.

Changes in TMRE uptake do not exclusively reflect altered membrane permeability as changes in the absolute number of mitochondria (a.k.a., mitochondrial mass) comprise a quantitative factor in mitochondria dynamics that can significantly influence TMRE results. Thus, to assess possible changes in mitochondrial mass, SCC9 cells were pulse-labeled with nonyl-acridine orange (NaO), which specifically labels the mitochondrial lipid cardiolipin, the levels of which are unchanged by fluctuations in membrane potential. In cells expressing ATF2^{T52E}, no changes in NaO uptake were observed compared with control cells. However, in cells expressing ATF2^{T52A}, increased NaO levels were observed, indicating that loss of mitochondrial membrane potential results in increased mitochondrial biogenesis, likely compensating for dysfunctional mitochondria (FIG. 11B, 11C), consistent with previous observations that genotoxic stress can stimulate mitochondrial biogenesis. Notably, the observed TMRE/NaO ratios, interpreted as membrane potential per mitochondria (or mitochondrial “leakiness”), indicate that overexpression of ATF2^{T52A}, but not ATF2^{WT} or ATF2^{T52E}, decreases membrane potential relative to control cells (FIG. 5C). Correspondingly, only the expression of T52A effectively caused leakage of cytochrome C (FIG. 5C, 12D). That ATF2 WT expression did not decreased mitochondrial potential or induced cell death (FIG. 5D) could be explained by its susceptibility to undergo T52 phosphorylation, which would result in its nuclear retention. Hence, conditions that only partially inhibit PKCε are expected to retain a portion of ATF2 nuclear, whereas 100% of the T52A is excluded from the nuclei. Indeed nuclear localization is seen in ˜40% of the ATF2^{WT} expressing cells (FIG. 11D), which may attenuate the effect elicited by the cytosolic ATF2. These observations suggest that a minimal level of ATF2 must reach the mitochondria in order to affect its membrane potential and support cell death.

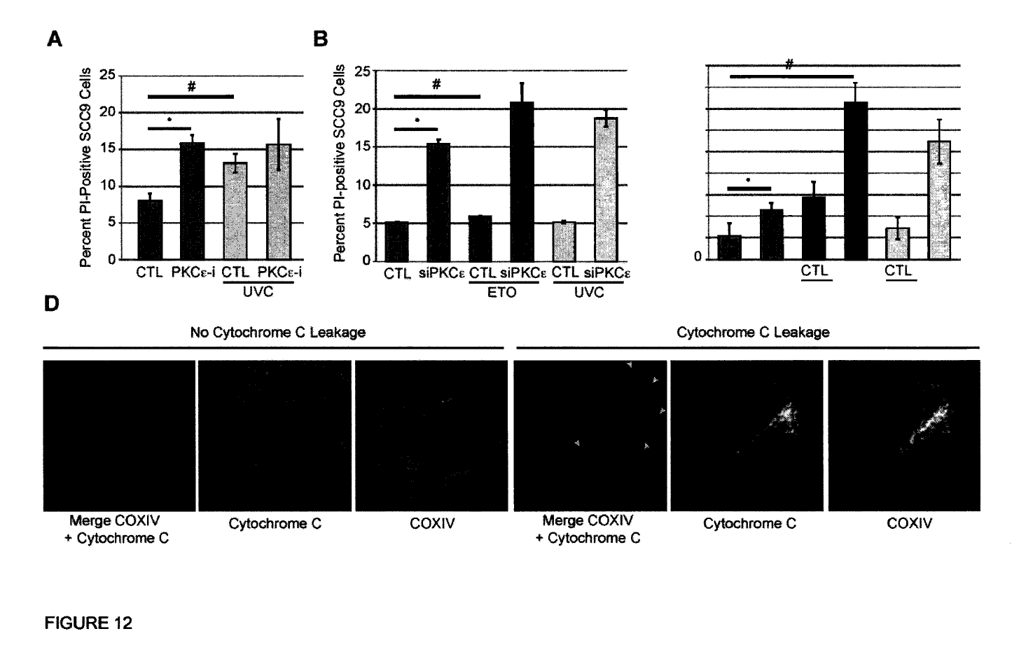

As mitochondrial leakage is often a precursor of cell death, it was hypothesized that mitochondria-localized ATF2 could sensitize cells to genotoxic stress-induced cell death. In order to test the hypothesis, the effect of PKCε inhibition of SCC9 cells exposed to genotoxic stress was evaluated. Cells treated with PKCε inhibitor exhibited increased Annexin-V labeling, a marker for early-phase cell death compared to control cells, similar to ETO-treated cells (FIG. 5E). The latter is consistent with the observation that PKCε activation was attenuated to similar levels by treatment with ETO or PKCε-i (FIG. 3D). Consistently, SCC9 subject to PKCε-i or siRNA-mediated knockdown of PKCε also exhibited increased cell death, as indicated by propidium iodide labeling, used here as a marker for late apoptosis, after etoposide or UVC (20 J/m²) treatment (FIG. 12A, 12B). In agreement, expression of ATF2^{T52A} but not WT or ATF2^{T52E} in SCC9 cells also resulted in elevated cell death, regardless of genotoxic stress (FIG. 5F).

### EXAMPLE 6

### PKCε Expression Increases T52 Phosphorylation of ATF2 and Resistance of Melanoma Cells to Genotoxic Stress

This example illustrates that PKCε phosphorylation of ATF2 on Thr52 increases resistance of melanoma cells to genotoxic stress-induced cell death. This example further illustrates that melanoma exhibiting upregulated PKCε are more resistant to genotoxic stress-induced cell death compared to lower expressing counterparts; ATF2 -T52E confers resistance to genotoxic stress.

To more directly investigate the PKCε phosphorylation of ATF2 on T52, a polyclonal antibody with specific affinity for phosphorylated T52 (pT52 antibody) was generated. Overexpression of caPKCε in SCC9 cells resulted in the appearance of a slower migrating band that was recognized by ATF2 antibody, and by the newly generated pT52 antibody (FIG. 6A). Both the total ATF2 and pT52 ATF2 bands were no longer seen upon inhibition of ATF2 expression by shRNA (FIG. 6B).

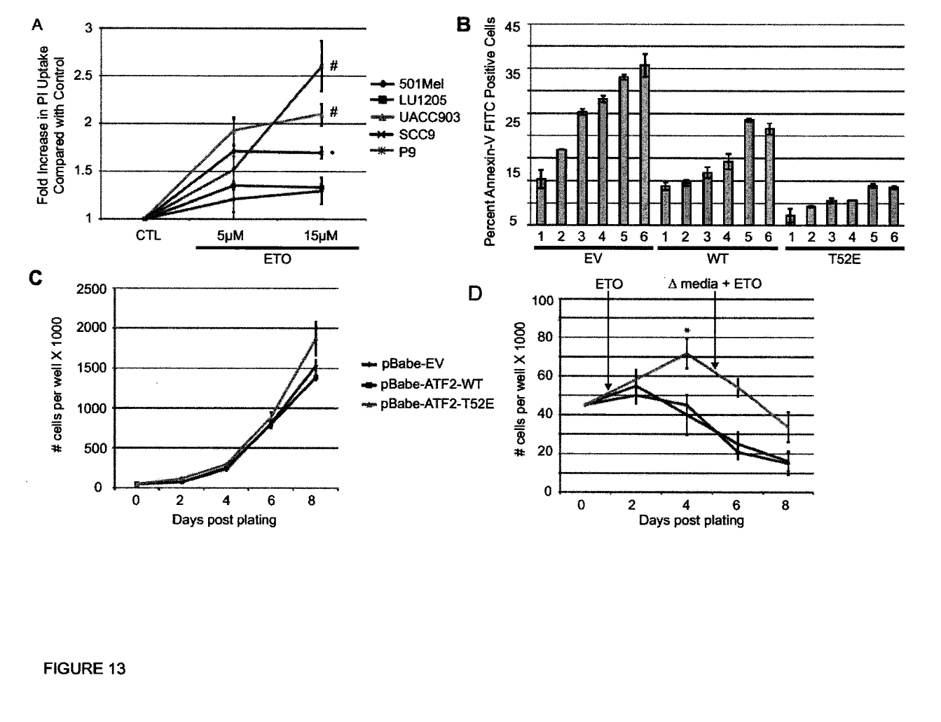

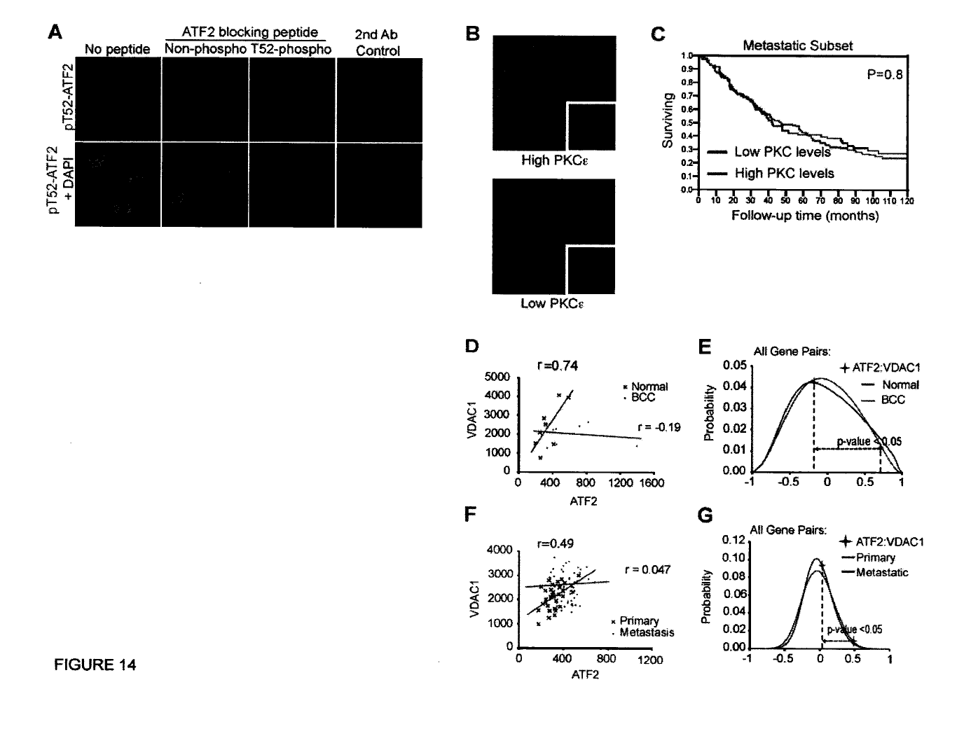

The status of pT52 in SCC9 was next evaluated, as well as melanoma cell lines UACC903, 501Mel and LU1205, prior and following genotoxic stress. Consistent with reduced PKCε phosphorylation of ATF2 on T52 after genotoxic stress, the levels of pT52 were lower in SCC9 cells subjected to etoposide treatment (FIG. 6C). Notably, decrease in T52 phosphorylation was also apparent in UACC903 cells, but to a much lesser degree in 501Mel and LU1205 cells. These findings are consistent with the relationship between PKCε activity and the ability of ATF2 to translocate to the mitochondria following genotoxic stress. While UACC903 exhibit mitochondrial translocation of ATF2 following etoposide treatment, similar to SCC9 cells, such translocation was not seen in the LU1205 or 501MEL cells (FIGS. 6C and D). Correspondingly, LU1205 and 501Mel cells were more resistant to cell death compared with UACC903, SCC9 or P9 (a squamous carcinoma cell line, where PKCε is also low, FIG. 13A), further supporting the notion that melanoma cells are more resistant to genotoxic stress-induced cell death due to impaired ATF2 translocation to mitochondria. Consistent with these observations and the localization observed by overexpression of the ATF2^{T52E} mutant, immunostaining with the pT52 antibody detected ATF2 that was restricted to the nucleus (FIG. 14A). These findings suggest that elevated PKCε levels in melanoma cells inhibit ATF2 localization and function at the mitochondria, thereby enhancing cell survival during genotoxic stress.

To further establish the relationship between PKCε activity, pT52 phosphorylation and susceptibility to undergo apoptosis following genotoxic stress PKCε or ATF2 in MEL501 and SCC9 cells were manipulated. Whereas MEL501 cells exhibit high level of PKCε, T52 phosphorylation (FIG. 6C, 7A) and impaired ATF2 translocation to the mitochondria (FIG. 6D), SCC9 have low level of PKCε, T52 phosphorylation (FIG. 6C, 7A) and exhibit ATF2 localization at the mitochondria following genotoxic stress (FIG. 1). When subjected to etoposide or UVC treatment in the presence of PKCε inhibitor or expression of siRNA targeted against PKCε, 501MEL were sensitized to genotoxic stress-induced cell death (FIG. 6E; 12C). Conversely, SCC9 cells expressing ATF2^{T52E} exhibited lower levels of Annexin-V labeling in response to etoposide, PKCε inhibitor, or a combination of both, in comparison to cells expressing empty vector (EV) or ATF2^{WT} (FIG. 13B), corroborating the pro-survival effects of T52 phosphorylation. Intriguingly, although the stable expression of either EV, ATF2^{WT} or ATF2^{T52E} in SCC9 cells exhibited no significant effects in proliferation rate during non-stressed conditions, ATF2^{T52E} conferred a survival advantage when the cells were grown in the presence of etoposide (FIG. 13C), further demonstrating the pro-survival effects conferred by pT52-ATF2.

### EXAMPLE 7

### High Expression of PKCε in Melanoma Tumors is Associated with Poor Prognosis

This example illustrates the clinical implications of PKCε expression from melanoma TMA, and bioinformatic expression correlation of ATF2 and VDAC in BCC and melanomas.

To determine the nature of the differences in genotoxic stress resistance between melanoma and SCC cells the degree of PKCε expression was assessed in addition to activity in melanoma cell lines and in human melanoma samples using a panel of melanoma cell lines and a large cohort melanoma tissue arrays (TMA).

Overall, melanoma cell lines exhibited higher PKCε expression than primary keratinocytes, melanocytes, or SCC lines (FIG. 7A). Furthermore, pT52 was notably lowest in primary keratinocytes and melanocytes, whereas its levels were highest in 501Mel and LU1205, where total and pS729 (active) PKCε levels are the highest.

Next, the association between PKCε expression levels and patient survival and other clinical and pathological factors were studied. Earlier studies had revealed that ATF2 subcellular localization in tumors is significantly correlated with prognosis: nuclear localization, reflecting constitutively active ATF2, was associated with metastastic tumors and poor outcome. Here PKCε expression was determined via immunofluorescence staining and independent scoring of the TMA. Staining within each histospot was fairly homogenous, revealing primarily cytoplasmic/membranous staining (FIG. 14B). Immunofluorescent intensity was measurable in 427 histospots (171 primary and 256 metastatic cases).

Expression of PKCε was higher in metastastic than primary specimens (P<0.0001), as shown in FIG. 7B. In primary specimens, PKCε levels were higher in thicker lesions (P<0.0001) and in ulcerated lesions (P=0.0004). No correlation was found between PKCε expression and age, gender or presence of tumor infiltrating lymphocytes.

By Cox univariate survival analysis of continuous immunofluorescent intensity scores, it was found that high PKCε expression was associated with decreased survival in the primary cohort (P=0.0042), but not in the metastatic subset (P=0.887). On multivariate analysis PKCε expression did not retain its independent prognostic value, presumably due to the strong association with Breslow Depth. To visually demonstrate the association between PKCε expression and survival, the continuous scores were dichotomized by the median score. High PKCε expression was not associated with survival in the metastatic subset of patients (log rank P=0.8; FIG. 14C), but in the primary cohort, high PKCε was strongly associated with decreased survival (log rank P=0.0004; FIG. 7C). These findings support earlier analyses of a similar cohort in which nuclear localization of ATF2 correlated with poor prognosis.

The analysis of a recent global mRNA expression profiling study revealed that expression of ATF2 and VDAC1 are highly correlated in non-malignant tissue samples, but that this correlation falls significantly in basal cell carcinoma (FIGS. 14D, E). An independent expression profiling study of metastatic versus non-metastatic melanoma also identified that ATF2 and VDAC1 expression levels were significantly more correlated in primary melanoma than in aggressive metastases (FIG. 14F, G). These analyses are consistent with ability to predict cancer outcome using dynamic modularity in protein interaction networks.

### EXAMPLE 8

### Experimental Procedures

### Cell Lines

Squamous cell carcinoma (SCC9 ) cells were maintained in Dulbecco's Modified Eagle's Medium (DMEM):F-12 (50:50) (Hyclone/Thermo Scientific, USA) supplemented with 400 ng/ml hydrocortisone, 10% fetal bovine serum (FBS) and antibiotics. Melanoma cell lines, M7, P9 and human skin fibroblasts were maintained in Dulbecco's Modified Eagle's Medium (DMEM) supplemented with 10% FBS and antibiotics. Normal human embryo kidney (NHEK) cells were maintained in KGM-2 growth medium (Lonza, USA).

Melanocytes were maintained in 254 Medium (Invitrogen, Calif., USA).

### Antibodies and Immunostaining Reagents

Antibodies employed were purchased as follows: ATF2 (20F1), HK1 (C35C4), HK2 (C64G5), HA (C29F4—for immunoblot analysis), COXIV (3E11) from Cell Signaling Technologies (MA, USA); β-actin (9), HIS-probe (H-15), HSP90 (4F10), Lamin A (N-16), PKCε (C15), TOM20 (F-10), VDAC1 (20B12) from Santa Cruz Biotechnology, Inc. (CA, USA); HA (3F10—for immunofluorescent staining) from Roche; COXI (21C11BC11), COXII (2E3), COX IV (20E8), pS729 PKCε (44977G) from Invitrogen (CA, USA); HSP60 (24) from BD Pharmingen, USA; pT52 ATF2 (PhosphoSolutions, Inc). MitoTracker Red CMXRos, PKCε translocation inhibitor, and Leptomycin B were purchased from Invitrogen (CA, USA), Santa Cruz Biotechnologies, (CA, USA), and Sigma (USA), respectively.

### Immunocytochemical Analysis

Following indicated treatments, coverslip-grown cells were fixed in fixation buffer containing 4% formaldehyde and 2% sucrose in phosphate buffered saline (PBS) for 20 minutes at room temperature. After fixation, coverslips were rinsed twice in PBS and permeabilized in permeabilization buffer (0.4% Triton X-100 (TX-100)/0.4% bovine serum albumin (BSA) in PBS) for 20 minutes. Primary antibodies were applied at 1:250 dilution in staining buffer (0.1% TX-100/0.1% BSA in PBS) overnight at 4° C. in a humid chamber. Coverslips were subsequently subject to 5 standing washes in wash buffer (0.2% TX-100/0.2% BSA in PBS, 5 minutes per wash). Secondary antibodies (Alexa Fluor secondary 350, 488 or 568, Invitrogen) were applied at 1:250 dilution in staining buffer for 2-3 hours at room temperature in a humid chamber in the dark. Prior to mounting with Vectorshield containing DAPI (Vector Laboratories, CA), coverslips were washed twice additionally in wash buffer. Immunofluorescent analysis was conducted on an Olympus TH4-100 fluorescent microscope. At least 3 Z-planes per field were captured and analyzed using Slidebook V.4.1, and Z-projection images of average intensity were constructed using ImageJ V.1.43u.

### EXAMPLE 9

### Development of HTS-Adaptable Assays and Screens to Identify Small Molecules that Induce Apoptosis in Melanoma Cells

This example describes methods of screening of small molecules that will modulate the ATF2 activity in melanoma. The example further illustrates the development of cell-based assays to identify small molecule drug candidates that will restore nuclear export of ATF2 and thereby sensitivity of melanoma to chemotherapy

The findings that cytosolic localization of ATF2, as seen following genotoxic stress, enables its localization at mitochondria to enhance apoptosis and that such localization is lost in malignant melanoma cells provide the foundation for a hypothesis that small molecules that mobilize ATF2 from the nucleus to the cytosol will sensitize melanoma cells to therapy. Such small molecules are expected to include inhibitors of PKCε and its upstream signaling components or factors functioning in nuclear export of ATF2. Given the notorious resistance of melanoma to therapy, the proposed studies establish a new paradigm for modulating transcription factor activity (via altered localization) as a means to sensitize chemotherapy resistant melanoma cells to treatment. A phenotypic pathway-based screen will be established and conducted to identify small molecules that would promote nuclear export and/or cytoplasmic/mitochondrial localization of ATF2 in melanoma cells bearing high levels of PKCε. Such inhibitors should sensitize melanoma cells to apoptosis following treatment with genotoxic agents, thereby offering a new therapeutic modality for metastatic, chemoresistant melanomas. Some potential inhibitors will likely be inhibitors of PKCε, a desirable target that could be explored in secondary and tertiary assays for new scaffolds with improved properties such as in-class selectivity.

Given that the ability to mobilize ATF2 to mitochondria offers a novel modality to sensitize metastatic melanoma cells to apoptosis, an unmet need in oncology will be met by the assays disclosed herein. In view of the existing barriers to monotherapy, the proposed approach offers the ability to interfere with a heretofore unrecognized signaling pathway in melanoma, ATF2 and its regulator PKCε. Notably, the availability of reagents to monitor ATF2 localization and function together with genetic and cell biological evidence for the role of ATF2 in melanoma provide an unparalleled opportunity to develop a novel therapy for future clinical trials.

The identification and development of small molecules to selectively affect ATF2 nuclear export and activate its tumor suppressor function differ from any existing therapy and therefore offer a novel mechanism of action that complements existing therapies in melanoma. Mobilization of ATF2 to the cytosol where it would alter mitochondrial permeability would inhibit ATF2 transcriptional activity and melanoma development. Additionally, a novel opportunity to target a transcription factor via a non-classical and arguably more tractable approach is provided. The robust secondary assay testing funnel and in vivo mouse model ensures the ability to advance lead compounds effectively.

HTS-adaptable assays will be developed and HTS screens conducted in order to identify small molecule leads that induce cytoplasmic relocalization of ATF2 and subsequent genotoxic stress-induced apoptosis in melanoma cells. Successful execution of these objectives should result in the identification of well-characterized compounds with ATF2 tumor suppressor-inducing activity that can then serve as starting points for medicinal chemistry. The availability of secondary assays, a genetic mouse melanoma model and antibodies/peptides to monitor ATF2 function in melanoma will ensure validation and subsequent characterization of expected hits from the proposed screen.

Develop/Validate High-Throughput Screen

The first stage of the project will involve optimization and validation of the high-content imaging assay in MeWO cells, chosen based on their high PKCε and lack of ATF2 translocation following genotoxic stress. An algorithm has been developed that facilitates the quantitative measurement of endogenous ATF2 subcellular localization in high-throughput format.

The existing plate preparation protocol will be modified and validated for use with automated liquid handlers/dispensers and if needed to accommodate a different cell line. DMSO tolerance of the cells will be evaluated to ensure that the assay can tolerate the DMSO concentrations encountered during the chemical library screen.

Following plate preparation optimization, image acquisition will be streamlined to speed-up imaging and the developed nuclear-to-cytoplasmic translocation assay read-out will be optimized and validated on the final assay conditions. The final assay protocol will then be validated using control compound dose response and screen concentration experiments to determine the false positive rate and assay performance (Signal-to Background ratio and Z′ value).

HTS Execution

Following successful optimization of the ATF2 localization assay, the next stage will be a high-content screen (HCS) for chemical inhibitors that are able to induce cytosolic ATF2 relocalization in melanoma cells in the presence of genotoxic stress. As a positive control a PKCε inhibitor will be used and the above described ATF2 50-100 peptide, both of which drive ATF2 nuclear exclusion. Image-based HCS (384-well format) will be conducted in the ATF2 localization assay, as developed above, against the entire SBMRI compound collection of 325,000 compounds, at a single concentration (10 μM). Screening a library of this size will maximize the chances of identifying viable and chemically tractable hits. Plate preparation and imaging will be performed in batches of 50-100 with 384-well plates. Additional read-outs generated for this assay will be used to flag any wells and/or assay plates with deviating cell counts or phenotypes. Data from HTS will be uploaded into the CBIS Database to enable data analysis and processing and generation of an initial hit list. Images for the initial hits as well as flagged wells will be visually evaluated to confirm the outcome. Multi-parametric analysis of the high-content image-data will be performed using Columbus, Spotfire and/or Genedata High-Content Analyzer software yielding plate quality control data as well as information on cytotoxic and autofluorescent effects of the compounds.

Primary hits will be reconfirmed in the same assay at single concentration in triplicate and simultaneously tested in a counter-screen for HSP60 localization to ensure changes in subcellular localization are ATF2-specific. Confirmed hits that do not affect HSP60 localization will be tested for their ability to induce apoptosis in chemotherapy-treated melanoma cells using the established Cytochrome C release assay. At this point, confirmed hits will be reordered as dry powders and retested in the primary ATF2 localization assay, the HSP60 counter screen as well as a several profiling assays described below that constitute the major SAR-driving assays.

Hit Validation/Secondary Screen

Following single-concentration confirmation in the ATF2 localization assay, hits will be tested in dose-response to establish potency (IC₅₀ values) in the HCS assay. Reconfirmed compounds will be evaluated for their ability to induce apoptosis as described see above in relevant cell types in an ATF2 -dependent fashion. In addition, compounds will be assessed for their ability to attenuate PKCε but not PKCδ activities in biochemical inhibition assays. Using the ATF2 subcellular localization assay, chemical compounds that would selectively inhibit PKCε, or its upstream regulators, as well as facilitate nuclear export of ATF2 will be identified. In addition, the studies described herein may result in the identification and characterization of compounds that selectively enable ATF2 to function at the mitochondria, thereby sensitizing melanoma cells to therapy induced apoptosis. Compounds that that act via these mechanisms will be further assessed for their (a) ability to cause cell death in melanoma cells (versus non-melanoma) prior to and following treatment with genotoxic stimuli by measuring cytochrome C release, annexin/PI staining, caspase 3/7 activation and overall viability; and (b) for their effect on ATF2 localization and cell death in melanocytes, keratinocytes and BCC/SCC cells, in which ATF2 is subject to such cytosolic translocation. In addition, compounds shown to be non-toxic to these control cultures will be advanced to the next stage of discovery to determine (c) effects on ATF2 transcriptional activity; and (d) their effect on PKCε and PKCδ activities. Since PKCδ antagonizes PKCε activity, only with inhibitors that demonstrate >10-fold selectivity of PKCε over PKCδ will be elected for further advancement. Active compounds will be further assessed in cytotoxicity assays using a panel of 15 melanoma cell lines representing different genetic mutations (B-Raf or N-Ras) and different metastatic potential. Compounds that exhibit the most potent effect in terms of promoting ATF2 nuclear export and tumor cell apoptosis following genotoxic treatment will be prioritized for medicinal chemistry efforts as well as future DMPK and xenograft studies. Combining newly discovered inhibitors with leading monotherapies, such as PLX4032, could also assessed in cell culture and in xenograft models.

### EXAMPLE 10

### Experimental Protocols for Assays to Determine Degree of Small Molecule-Mediated Melanoma Cell Sensitization to Apoptosis

The example describes a procedure for the optimization and validation of high-content imaging assay in MeWO cells, chosen based on their high PKCε and lack of ATF2 translocation following genotoxic stress. This example illustrates the development of an algorithm that facilitates the quantitative measurement of endogenous ATF2 subcellular localization in high-throughput format.

### Primary and Validation Assays

Immunocytochemical Analysis

Melanoma cells that express high level of PKC and render ATF2 nuclear even following genotoxic stress will be used. Among these cultures are 501Mel, WM793 and MeWo. Cells are maintained in Dulbecco's Modified Eagle's Medium (DMEM) supplemented with 10% FBS and antibiotics. HT cell based assays will be conducted in 384 wells plates. Cells will be subjected to treatment with the small molecule library in the presence of genotoxic stress, followed by fixation (4% formaldehyde and 2% sucrose in PBS for 20 minutes at room temperature) 12-24 hours later. Cells are than rinsed twice in PBS and permeabilized (0.4% Triton X-100/0.4% BSA in PBS) for 20 minutes. Monoclonal antibodies to ATF2 (raised in-house) will be used (diluted in 0.1% TX-100/0.1% BSA in PBS) to monitor endogenous ATF2 localization. As negative control, localization of HSP60 and p53 will be monitored using commercially available antibodies. P53, which is translocated to the mitochondria following genotoxic stress is not dependent on PKC and hence modulators of PKC or ATF2 should not affect p53 translocation. ATF2-Alexa 488 (384-well format) will be imaged with the Opera QEHS system using DAPI and AlexaFluor 488 wavelengths. Quantification of nuclear metrics from nuclei regions of DAPI images and ATF2-based nuclei and cytoplasmic region metrics from ATF2-Alexa 488 images will be made on a cell-by-cell level. Image analysis will be performed using Acapella/Columbus HCS image analysis software. The possibility to engineer melanoma cells that stably express RFP-ATF2, thereby enabling to simplify the screen by alleviating the need for immunostaining will be considered at the outset. HSP60 staining, for example, can be used as a mitochondrial localized protein marker.

### Secondary Assessment

Sensitization of Resistant Melanoma Cells to Apoptosis

Melanoma cells resistant to treatment by genotoxic agents became sensitive following inhibition of PKCε, or expression of mutant ATF2 on T52 following treatment with etoposide or UV irradiation in the presence or absence of PKCε siRNA). Small molecules identified as capable of mobilizing ATF2 from the nucleus to the cytosol are expected to sensitize melanoma cells to apoptosis.

Among the primary surrogate assays to determine degree of such sensitization will be monitoring degree of apoptosis elicited by positive hits identified and confirmed in primary assays, when added alone or in combination with genotoxic agent. ATPlite, cytochrome C and caspase 3/7 staining has been extensively used in previous studies and will be used for this initial validation.

### Tertiary Assessment

Measurement of Mitochondrial Membrane Potential.

A surrogate assay for the effectiveness of small molecules that causes ATF2 nuclear export is the assessment of altered mitochondrial potential, with and without exposure to genotoxic stress. Preliminary results establish the effect of ATF2 nuclear exclusion on mitochondrial membrane permeability. For this purpose melanoma cells were pulse labeled with 300 nM tetramethylrhodamine ethyl ester (TMRE; Invitrogen, Calif.) followed by FACS analysis analyzed by FlowJo software. In the proposed studies, to enable HTP analysis of selected hits, the same TMRE uptake assay in multi-well plates will be used, quantitated by luminometer.

Analysis of ATF2 Transcriptional Activity

A decrease in ATF2's transcriptional activity is a surrogate readout for its nuclear exclusion. Corresponding luciferase assays will be performed on positive hits. Melanoma cells plated in 96-well plates (3,000 cells/well) will be established to stably express pCMV β-galactosidase, and pGL-Jun2-promoter construct. Small molecules selected from the initial screen will be added and cells will be assessed for luciferase activity using the Luciferase Assay System (Promega, Wis.), normalized by standardized β-gal assay.

Inhibition of PKCε Phosphorylation

A tertiary analysis for hits that withstand the secondary assessment would include evaluation of PKC epsilon versus PKC delta activity. Initial analysis for PKC epsilon activity would come by analysis of ATF2 phosphorylation on T52. Select small molecules that were found to specifically affect ATF2 translocation, and which effectively sensitize melanoma cells to apoptosis will be assessed by pAb raised against phosphorylated form of T52. Currently, the pT52 antibodies are used in westerns, and their evaluation for use in IF and IHC is ongoing. Further, as it is of importance to determine the effect of these select small molecules on inhibition of PKCε but not PKCδ their phosphorylation on residues required for their activity will be monitored. PKCδ phosphorylation on S729 and PKCδ phosphorylation on amino acid T505 are associated with their activation. Commercially available antibodies (Invitrogen and Cell Signaling, respectively) will allow monitoring of changes on these phosphorylation sites following treatment of melanoma cells with the small molecules that passed the primary and secondary assessments. Additional analysis will be performed using kinase reactions with commercially available active kinase, on respective substrates (Millipore). Small molecules that selectively inhibit PKCε but not PKCδ would be rendered valuable for applications in other cancers where PKCε has been documented to function as oncogene, thereby providing long sought solution to highly desired clinical target.

Although the invention has been described with reference to the above example, it will be understood that modifications and variations are encompassed within the spirit and scope of the invention. Accordingly, the invention is limited only by the following claims.

What is claimed is:

1. A method for the diagnosis or prognosis of a cellular proliferative disorder in a subject comprising:
obtaining from the subject a biological sample; and
determining in the sample the expression level of PKC, wherein an increased level of PKC expression in the sample compared to a healthy control indicates a presence or an increased likelihood of a cellular proliferative disorder, thereby providing a diagnosis or prognosis of the disorder in the subject.

2. The method of claim 1, wherein the disorder is a melanocyte proliferative disorder.

3. The method of claim 1, further comprising:
obtaining from a subject suffering from a cellular proliferative disorder a pre-therapeutic treatment sample and a post-therapeutic treatment sample; and
determining the expression level of PKC in the samples, wherein a decreased level of PKC in the post-treatment sample compared to the pre-treatment sample is indicative of a positive therapeutic treatment.

4. A method of treating a cellular proliferative disorder in a subject comprising administering to the subject an effective amount of an agent that modulates translocational activity of ATF2, thereby treating the disorder.

5. The method of claim 4, wherein the agent promotes or increases the translocational activity of ATF2.

6. The method of claim 4, wherein the translocational activity is nuclear export or nuclear exclusion.

7. The method of claim 4, wherein the translocational activity is cytosolic localization.

8. A method of treating a cellular proliferative disorder in a subject comprising administering to the subject an effective amount of an agent that modulates activity of PKC, thereby treating the disorder.

9. The method of claim 8, wherein the agent inhibits expression of PKC.

10. The method of claim 8, wherein the agent is a selective inhibitor of PKCε.

11. The method of claim 10, wherein the PKCε selective inhibitor has a selectivity ratio of PKCδ IC₅₀ to PKCε IC₅₀ not less than about 10.

12. The method of claim 4, wherein the disorder is a melanocyte proliferative disorder.

13. The method of claim 12, wherein the disorder is melanoma.

14. The method of claim 13, wherein the disorder is metastatic melanoma.

15. The method of claim 12, further comprising administering vemurafenib (Zelboraf™).

16. A method for identifying an agent for sensitizing cancer cells or tumor cells to apoptosis comprising:
contacting cancer cells or tumor cells with a test agent; and
detecting apoptosis induced in the cancer cells or tumor cells contacted with the agent as compared to cells not contacted with the agent, wherein apoptosis in the cells contacted with the agent is indicative of an agent that sensitizes cancer cells or tumor cells.

17. The method of claim 16, wherein prior to, simultaneous with or following contacting the cells with the test agent, the cells are subjected to genotoxic stress.

18. The method of claim 17, wherein the genotoxic stress is chemotherapy, radiation therapy, or phototherapy.

19. The method of claim 16, wherein the tumor cells are melanocytes.

20. A method for the diagnosis of or prediction of risk of a cellular proliferative disorder in a subject comprising:
obtaining a biological sample from the subject;
contacting the sample with an antibody that recognizes phosphorylated ATF2; and
detecting the level of phosphorylated ATF2, wherein an increased level of phosphorylated ATF2 in the sample compared to a healthy control indicates a presence of or increased risk of a cellular proliferative disorder, thereby providing a diagnosis of the disorder in the subject.

21. The method of claim 20, wherein the phosphorylated ATF2 is phosphorylated at position T52.

22. The method of claim 20, wherein the phosphorylated ATF2 is primarily in the nucleus.

23. The method of claim 20, wherein an increased level of phosphorylated ATF2 in the nucleus is indicative of a likelihood of resistance of the cell to genotoxic stress and cell survival.

24. An antibody that specifically binds to phosphorylated ATF2.

25. The antibody of claim 24, wherein the phosphorylation is at T52.

26. The antibody of claim 24, wherein the antibody is polyclonal.

27. The antibody of claim 24, wherein the antibody is monoclonal.
